(19) United States
(12) Patent Application Publication
(10) Pub. No.: US 2022/0226499 A1
(43) Pub. Date: Jul. 21, 2022

(54) Metabolite Delivery for Modulating Metabolic Pathways of Cells

(71) Applicant: Arizona Board of Regents on Behalf of Arizona State University, Scottsdale, AZ (US)

(72) Inventor: Abhinav Acharya, Tempe, AZ (US)

(21) Appl. No.: 17/611,758

(22) PCT Filed: May 15, 2020
(86) PCT No.: PCT/US20/33144

§ 371 Date: Nov. 16, 2021

Related U.S. Application Data
(60) Provisional application No. 62/848,682, filed on May 16, 2019.

(51) Int. Cl. A61K 47/69; A61K 31/194; A61K 47/55; A61K 9/51; A61L 24/04; A61L 27/34; A61L 27/52; A61L 31/10

(52) U.S. Cl. CPC A61K 47/6929; A61K 31/194; A61K 47/55; A61K 47/6927; A61L 31/10; A61L 24/046; A61L 27/34; A61L 27/52; A61K 9/5146

(57) ABSTRACT

The present disclosure provides metabolite-based polymeric particles and methods for a modulating the intracellular metabolic-profile/pathways. For example, in one aspect, the disclosure relates to alpha-ketoglutarate (aKG)-based polymeric-microparticles and methods of use.

### CROSS-REFERENCE TO RELATED APPLICATIONS

The present application claims priority to U.S. Provisional Application Ser. No. 62/848,682, filed May 16, 2019, which is incorporated by reference herein in its entirety.

### BACKGROUND

There is a rich history of successful drug delivery carriers made of biodegradable biomaterials that can modulate immune responses. Examples of such carriers include polyesters (e.g. poly (lactic-co-glycolic) acid (PLGA)—used in applications ranging from cancer to autoimmunity), and bi-lipid layer carriers (e.g. liposomes). Notably, these biomaterials degrade into metabolic by-products, which are capable of modulating the function of immune cells. For example, the degradation product of the drug delivery carrier poly(lactic acid) is lactic acid (a by-product of glycolysis), which can directly suppresses immune cells such as dendritic cells, (DCs—specialized immune cells responsible for inducing adaptive immune responses), macrophages (phagocytes, responsible for removing debris) and T-cell lymphocytes (responsible for mounting immune responses against foreign materials). Interestingly, there are several metabolites that are known to modulate function of immune cells including, succinate—activates DCs and lead to adaptive immune response, citrate—induces pro-inflammatory cytokines and reactive oxygen species, α-ketoglutarate—induces alternate activation (immunosuppressive phenotype) in macrophages through metabolic reprogramming, and polyunsaturated fatty acids (e.g. arachidonic acid C20:4(n-6))—blocks activation of DCs.

There is a great need to modulate the metabolism of immune cells, which controls their function including inflammation, suppression and tolerance. However, currently there are no methods to deliver these metabolites intracellularly in these cells, without modifying the metabolite itself.

Immunometabolism reprogramming is an emerging and exciting new field that is involved in the induction, progression, and therapy of several diseases such as cancer, infections, autoimmune disorders, and Alzheimer's among others. Notably, modulation of immunometabolism can be performed by delivery of cell permeable metabolites (e.g., 2-hydroxyglutarate or enzymatic inhibitors (e.g., 2-deoxyglucose). For example, regulatory T cell (Treg—immunosuppressive) and T-helper type 17 (Th17—pro-inflammatory) differentiation can be controlled by modulating the glutamate oxaloacetate transaminase 1 (GOT1) enzyme, which has direct implications in immunosuppressive applications. On the other hand, metabolites provided via dietary interventions may improve immune cell function in pro-inflammatory applications such as cancer. Notably, increasing energy production from the Kreb's cycle without affecting glycolysis can reduce the function of pro-inflammatory effector T-cells, without affecting the function of regulatory anti-inflammatory Tregs (required for regeneration). These strategies can be targeted toward the adaptive branch of the immune system to generate effector function. Interestingly, dendritic cells (DCs) that form the bridge between innate and adaptive immune responses, are capable of inducing robust adaptive and innate immune responses, which is advantageous in diseases such as cancer, infections, and wound healing.

Interestingly, DCs play an important role in wound healing, potentially by secreting growth factors and cytokines important for the proliferation phase required for wound closure. Therefore, targeting DCs that can not only modulate the innate cells (e.g., neutrophils) in the wound bed, but also adaptive cells (e.g. regulatory T cells) can greatly accelerate wound healing responses. Importantly, activated immune cells (e.g., macrophages type 1, activated DCs, and Th1) have an enhanced glycolysis profile in various pro-inflammatory environments, including wound beds, which hampers faster closure responses. Therefore, modulating the energy-metabolic pathways of the immune cells can be a viable strategy for controlling macrophages and DC responses and effect the immune responses within the wound bed.

Although metabolites control immune cell functions, current approaches are unable to deliver these metabolites locally and intracellularly in a sustained manner.

Thus, there is a need in the art for compositions and methods modulating the intracellular metabolic profile of phagocytes for treating specific immune-related diseases, rather than the currently used approach of utilizing conventional drug carriers non-discriminably for all types of diseases. The present disclosure satisfies this need.

### SUMMARY OF THE INVENTION

In one aspect, a particle comprising a polymer of a metabolite is provided. In one embodiment, the metabolite comprises a phosphate group or a carboxylic acid group. In one embodiment, the metabolite is selected from the group consisting of α-ketoglutarate, succinic acid, Fructose, 1, 6 biphosphate (F16BP), fructose 6 phosphate, and phosphoenol pyruvic acid, and ribose 6 phosphate.

In one embodiment, the metabolite is α-ketoglutarate. In one embodiment, the polymer of α-ketoglutarate comprises a structure of formula (I):

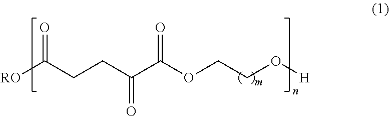

wherein R is a group selected from the group consisting of hydrogen, halogen, alkyl, aryl, heteroaryl, cycloalkyl, heterocyclyl, and combinations thereof; m is an integer from 1 to 30; and n is an integer greater than or equal to 1.

In one embodiment, particle is a microparticle. In one embodiment, the particle has a molecular weight of about 1 kDa to about 25 kDa. In one embodiment, the particle encapsulates an active agent.

In one embodiment, a biomaterial is provided. In one embodiment, the biomaterial is coated with a composition of the disclosure. In one embodiment, biomaterial is an implant, an adhesive.

In one embodiment, the disclosure provides a composition comprising a particle of the disclosure. In one embodiment, the disclosure provides a pharmaceutical composition comprising a particle of the disclosure. In one embodiment, the pharmaceutical composition is formulated for oral delivery, topical delivery, subcutaneous delivery, or intravenous delivery.

In one aspect, the disclosure provides a method of modulating the intracellular metabolite profile of a dendritic cell. In one embodiment, the method comprises administering a particle comprising a polymer of a metabolite to the subject. In one embodiment, the particle delivers the metabolite to the subject and modulates the intracellular metabolite profile of one or more dendritic cells in the subject.

In one embodiment, the method modulates the glutamate pathway of the dendritic cell. In one embodiment, the method increases one or more of L-glutamate, 4-aminobutanoate and asparagine in the cell. In one embodiment, the method modulates the Krebs cycle pathway of the dendritic cell. In one embodiment, the method modulates the glycolysis pathway of the dendritic cell. In one embodiment, the method modulates the arginine pathway of the dendritic cell. In one embodiment, the method increases one or more of aspartate, acetyl-ornithine and L-citrulline in the cell.

In one aspect, the disclosure provides a method of modulating immune response in a subject. In one embodiment, the method comprises administering a particle comprising a polymer of a metabolite to the subject, wherein the particle delivers the metabolite to the subject. In one embodiment, the method decreases pro-inflammatory T cell responses in the subject. In one embodiment, the method rescues immune cells against metabolic exhaustion.

In one aspect, the disclosure provides a method of treating a disease or disorder in a subject. In one embodiment, the method comprises administering a particle comprising a polymer of a metabolite to the subject, wherein the particle delivers the metabolite to the subject. In one embodiment, disease or disorder is associated with increased immune activation.

In one aspect, the disclosure provides a method of forming a metabolite-based polymer. In one embodiment, the method comprises forming a mixture of the metabolite comprising a carboxylic group and a diol compound of formula (A):

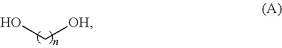

wherein n is an integer from 2 to 30. In one embodiment, n is 4, 6, 8 or 10. In one embodiment, the method further comprises heating the mixture at about 35° C. to about 200° C. In one embodiment, the mixture is heated for about 30 minutes to about 72 hours. In one embodiment, the disclosure provides a polymer formed by the methods of the disclosure.

### BRIEF DESCRIPTION OF THE DRAWINGS

The following detailed description of preferred embodiments of the disclosure will be better understood when read in conjunction with the appended drawings. For the purpose of illustrating the disclosure, there are shown in the drawings embodiments which are presently preferred. It should be understood, however, that the invention is not limited to the precise arrangements and instrumentalities of the embodiments shown in the drawings.

FIG. 1 depicts a schematic of one of the applications of this technology in wound healing.

FIG. 2, comprising FIG. 2A through FIG. 2D, depicts experimental results demonstrating that polymers can be generated from central-carbon metabolites as monomers. FIG. 2A depicts a reaction scheme of generating PaKG is shown. FIG. 2B depicts a 1H NMR demonstrating the generation of PaKG polymer. FIG. 2C depicts a scanning electron microscope image showing the size and morphology of PaKG particles. FIG. 2D depicts dynamic light scattering of particles showing the average size of the PaKG particles.

FIG. 3 depicts the characterization of the size and morphology of particles.

FIG. 4 depicts experimental results demonstrating that polymers of central metabolites differentially modulate the IL-10 production in DCs.

FIG. 5 depicts experimental results demonstrating that dendritic cells are able to phagocytose polyethyleneglycolsuccinate (PEGS) and PaKG particles encapsulating fluorescent molecules (red).

FIG. 6 depicts experimental results demonstrating that metabolite-based polymers upregulate activation markers in DCs differentially as observed by the MHCII and CD86 expression using flow cytometry.

FIG. 7 is a schematic demonstrating central-carbon metabolite-based polymers modulate adaptive immune responses by changing the intracellular metabolites of the dendritic cells.

FIG. 8, comprising FIG. 8A through FIG. 8D, depicts experimental results demonstrating central-carbon metabolite-based microparticles release metabolites in a sustained manner. FIG. 8A depicts a schema of PaKG synthesis. FIG. 8B depicts a scanning electron microscope micrograph of PaKG microparticles (MPs) (scale bar=5 μm). FIG. 8C depicts the dynamic light scattering size distribution of PaKG microparticles (average=4000 nanometer). FIG. 8D depicts the cumulative release kinetics of alpha-ketoglutarate (aKG) from PaKG MPs determined via high performance liquid chromatography (n=3, avg±SEM).

FIG. 9, comprising FIG. 9A through FIG. 9I, depicts experimental results demonstrating PaKG microparticles affect function of dendritic cells by modulating their metabolism. FIG. 9A depicts a fluorescent micrograph demonstrating bone marrow-derived dendritic cells (BMDCs) are able to phagocytose PaKG microparticles, (PaKG MP—magenta, cytosol—green, nucleus—blue; scale bar=70 μm). FIG. 9B depicts experimental results demonstrating PaKG MPs modulate intracellular metabolite levels as observed by significant (p<0.05) changes in 113 metabolites (out of 299 analyzed using LC-MS/MS), and their respective signaling pathways (1: Glycine/Serine/Threonine; 2: Arginine biosynthesis; 3: Glyoxylate metabolism; 4: Glutamate metabolism; 5: Arginine metabolism; 6: Glutathione metabolism.) The Pathway impact is number of metabolites modified significantly in a pathway; log(p) is the level of modulation. FIG. 9C and FIG. 9D depict experimental results demonstrating oxygen consumption rate (OCR) of DCs is reduced when cultured with PaKG MPs in the presence or absence of LPS. FIG. 9C depicts experimental oxygen consumption rate of DCs when cultured with PaKG MPs absence of LPS. (*, #−p<0.05, *−PaKG MPs significantly different than soluble aKG and no treatment; #−PaKG MPs significantly different than soluble aKG). FIG. 9D depicts experimental oxygen consumption rate of DCs when cultured with PaKG MPs absence of LPS (*−p<0.05; all groups significantly different than each other). FIG. 9E depicts the maximal respiration and spare capacity of DCs when cultured with PaKG MPs. FIG. 9F depicts the glycolysis of DCs in the presence of PaKG MPs (n>10, avg±SEM, *−p<0.05). FIG. 9G depicts the activation of DCs in the presence of PaKG MPs as indicated by frequency of MHCII⁺CD86⁺ in CD11c⁺ cells. (n>5, avg±SEM, *−p<0.05). FIG. 9H depicts the ratio of anti-inflammatory cytokine IL-10 to pro-inflammatory TNF-alpha in the presence of PaKG MPs (n=6, avg±SEM, *−p<0.05—a: LPS, PaKG; b: No treatment, PaKG MPs, PaKG MPs+LPS; c—No treatment, PaKG MPs, PaKG MPs+LPS; d: PaKG MPs, PaKG MPs+LPS; e: all conditions, f: LPS, PaKG MPs, soluble aKG, soluble aKG+LPS). FIG. 9I depicts the ratio of anti-inflammatory cytokine IL-10 to pro-inflammatory IL-12p70 in the presence of PaKG MPs (n=6, avg±SEM, $−p<0.05—significantly different than LPS alone and PaKG in absence of LPS; all other conditions are not significantly different from one another). Please see Example 2 for more details.

FIG. 10, comprising FIG. 10A through FIG. 10E depicts experimental results demonstrating PaKG microparticles modulate allogeneic adaptive immune responses in vitro FIG. 10A is a schematic of flow plot analysis. FIG. 10B depicts T helper type 1 cell frequency (Th1). FIG. 10C depicts T helper type 17 cell frequency (Th17). FIG. 10D depicts T helper type 2 cell frequency (Th2). FIG. 10E depicts regulatory T cell frequency (Treg). (n=6, avg±SEM, *−p<0.05—significantly different than no treatment control).

FIG. 11, comprising FIG. 11A through FIG. 11E, depicts experimental results demonstrating PaKG microparticles (100 mg/kg) when applied directly on the wound (5 mm initial diameter) accelerate wound healing responses. FIG. 11A depicts the timeline of the in vivo experiments in BALB/c mice. FIG. 11B depicts representative images of the wound after treatment with PaKG microparticles demonstrate faster wound healing as compared to the other groups. (Note—the dark region represents scab and closed wound in PaKG microparticles group) (n=6, avg±SEM, *−p<0.05). FIG. 11C depicts the wound area (normalized to day 0), which demonstrates that the PaKG microparticles are able to close the wound within 10 days (n=6, avg±SEM, *−p<0.05, significantly different than all other groups). FIG. 11D depicts the ultimate tensile strength (UTS) and % strength as compared to the intact skin was found to be highest for PaKG microparticles and lowest for soluble aKG group (n=6, avg±SEM, *−p<0.05, significantly different than all other groups). FIG. 11E depicts PaKG microparticles modulate wound healing by modulating the proliferating Th1 (pTh1) cell population in the skin, Th2 cell population in the draining lymph nodes, and lowered T cell responses systemically in spleen. (n=4, normalized to PBS, avg±SEM, *−p<0.05). Values normalized to PBS control, and numbers indicate fold increase or decrease.

FIG. 12 is a ¹H NMR spectrum of PaKG polymer. The ¹H NMR spectra demonstrates that the polymer was generated with aKG and 1, 10-decanediol as monomers.

FIG. 13 depicts experimental results demonstrating the molecular weight determination of the PaKG polymer. Method I—Mn and Mw are calculated using a calibration curve generated from polystyrene standards 500 KDa, 200 KDa, 100 KDa, 30 KDa, 10 KDa and 5 kDa, obtained from Agilent). Method II: Mw is calculated by determining the refractive index increment (dn/dc) using the refractive index detector and the assumption of 100% recovery, then using the light scattering detector response to determine an absolute molecular weight. Method III: Mn based on calculation of degree of polymerization using integrations from the ¹H NMR spectrum

FIG. 14 depicts experimental results demonstrating Glutamate and Arginine pathways are significantly upregulated in DCs treated with PaKG MPs as compared to no treatment (n=3, avg±SEM, *−p<0.05).

FIG. 15 depicts representative images of analyses of T-cells and DCs using flow cytometry analyses.

FIG. 16 depicts experimental results demonstrating individual components do not modulate the activation of DCs in vitro. (n=6, avg±SEM, ns=not significant).

FIG. 17 depicts experimental results demonstrating PaKG microparticles do not modulate frequency of CD4 population in allogenic MLR (ns—not significant; n−6, avg±SEM).

FIG. 18 depicts experimental results demonstrating PaKG MPs show a lower trend of DC activation in the skin as compared to the soluble aKG control. (n=2-4, avg±SEM, no groups significantly different than each other).

FIG. 19 is a schematic demonstrating alpha-ketoglutaric acid-based polymers induce immune suppression by modulating metabolism of dendritic cells.

### DETAILED DESCRIPTION

The present disclosure is based, in part, on the development of novel active metabolite-based polymeric particles. In one aspect, the disclosure provides compositions and methods for a modulating the intracellular metabolic-profile/pathways of dendritic cells (DCs). In one aspect, the disclosure relates to metabolite-based polymeric particles. For example, in one aspect, the disclosure provides alpha-ketoglutarate (aKG)-based polymeric-microparticles. These particles, when administered to a subject, provide sustained release of aKG and promote an immunosuppressive cellular phenotype.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “nanoparticle” refers to a particle having at least one dimension in the range of about 1 nm to about 1000 nm, including any integer value between 1 nm and 1000 nm (including about 1, 2, 5, 10, 20, 50, 60, 70, 80, 90, 100, 150, 200, 250, 300, 400, 500, 600, 700, 800, 900 and 1000 nm and all integers and fractional integers in between). In some embodiments, the nanoparticle has at least one dimension, e.g., a diameter, of about 100 nm. In some embodiments, the nanoparticle has a diameter of about 200 nm. In other embodiments, the nanoparticle has a diameter of about 500 nm. In yet other embodiments, the nanoparticle has a diameter of about 1000 nm (1 μm). In such embodiments, the particle also can be referred to as a “microparticle.” The particles can have any shape. Nanoparticles having a spherical shape are generally referred to as “nanospheres.”

As used herein, “microparticle” refers to a particle having at least one dimension in the range of about 1 μm to about 100 μm, including any integer value between 1 μm and 100 μm (including about 1, 2, 5, 10, 20, 30 40, 50, 60, 70, 80, 90 and 100 μm and all integers and fractional integers in between). Exemplary microparticles have a diameter of less than about 100 microns, less than about 50 microns, less than about 10 microns, less than about 5 microns, or less than about 3 microns, or less than about 2 microns. The particles can have any shape. Microparticles having a spherical shape are generally referred to as “microspheres.” The term “particle” as used herein is meant to include nanoparticles and microparticles.

As used herein, the term “treating” means ameliorating the effects of, or delaying, halting or reversing the progress of a disease or disorder. The word encompasses reducing the severity of a symptom of a disease or disorder and/or the frequency of a symptom of a disease or disorder.

As used herein, a “prophylactic” or “preventive” treatment is a treatment administered to a subject who does not exhibit signs of a disease or disorder or exhibits only early signs of the disease or disorder for the purpose of decreasing the risk of developing pathology associated with the disease or disorder.

As used herein, a “therapeutic” treatment is a treatment administered to a subject who exhibits signs of pathology of a disease or disorder for the purpose of diminishing or eliminating those signs.

As used herein, the term “subject” refers to a human or another mammal (e.g., primate, dog, cat, goat, horse, pig, mouse, rat, rabbit, and the like). In many embodiments of the present disclosure, the subject is a human being. In such embodiments, the subject is often referred to as an “individual” or a “patient.” The terms “individual” and “patient” do not denote a particular age.

“Molecular weight” as used herein, generally refers to the molecular weight as determined by gel permeation chromatography (GPC) unless otherwise specified. In practice, molecular weight can be estimated or characterized using various methods including gel permeation chromatography (GPC), the relative average chain length of the bulk polymer, or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% of the distribution lies within 15% of the median particle size, more preferably within 10% of the median particle size, most preferably within 5% of the median particle size.

“Active Agent”, as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

The term “biomaterial, as used herein, means a material that is biocompatible with a human or animal body. The biomaterial may be a a natural or synthetic biocompatible material suitable for introduction into living tissue. Natural biomaterials are materials made by biological systems. Synthetic biomaterials are materials that are not made in biological systems. The biomaterial disclosed herein may be natural biomaterials, synthetic materials or a combination of natural and synthetic biomaterials. Biomaterials used herein include, for example, polymer matrices and scaffolds.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

As used herein, “glycol” refers to a glycol compound having up to 24 carbon atoms, including but not limited to ethylene glycol, propylene glycol, butylene glycol, isobutylene glycol, hexylene glycol, or dodecanediol. means.

Ranges: throughout this disclosure, various aspects of the disclosure can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Metabolite Polymeric Particles

The present disclosure is based, in part, on the development of novel active metabolite-based polymeric particles. In one embodiment, the metabolite-based polymeric particles are capable of modulating the intracellular metabolic profile of phagocytes for treating specific immune-related diseases. In one embodiment, the metabolite-based polymeric particles are capable of modulating the glutamate pathway, the arginine pathway or both. In one embodiment, the metabolite-based polymeric particles are capable of promoting wound healing.

In one embodiment, the particle is a microparticle or nanoparticle. In one embodiment, the particle is a microparticle.

In one embodiment, the particle comprises a polymer of a metabolite. In one embodiment, the metabolite comprises a phosphate group or a carboxylic group. In one embodiment, the metabolite is α-ketoglutarate, succinic acid, glutamic acid, fructose 1, 6 biphosphate (F16BP), fructose 6 phosphate, phosphoenolpyruvic acid, acetyl coenzyme A, citric acid, fumarate, DL-Isocitric acid, malic acid, oxaloacetic acid, sodium pyruvate, succinyl coenzyme A, sodium succinate, and α-Ketoglutaric acid glucosamine, glutamine, glutamate, glucosamine 6P, N-Acetylglucosamine, UDP-GlcNAc, acetoacetate, beta-hydroxybutyrate, malonyl-CoA, phospholipids, serotonin, melatonin, or cystathionine.

In one embodiment, the particle comprises a polymer of α-ketoglutarate. In one embodiment, the polymer of α-ketoglutarate comprises a structure of formula (1):

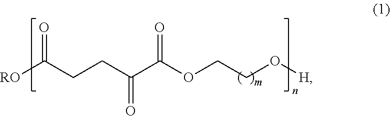

wherein R is a group selected from the group consisting of hydrogen, halogen, alkyl, aryl, heteroaryl, cycloalkyl, heterocyclyl, and combinations thereof; m is an integer from 1 to 30; and n is an integer greater than or equal to 1.

In one embodiment, the polymer of α-ketoglutarate comprises the structure,

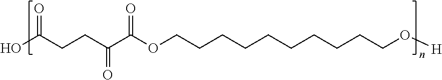

wherein n is an integer greater than or equal to 1.

In one embodiment, the particle comprises a polymer of F16BP. In one embodiment, the polymer of F16BP comprises a structure of formula (2):

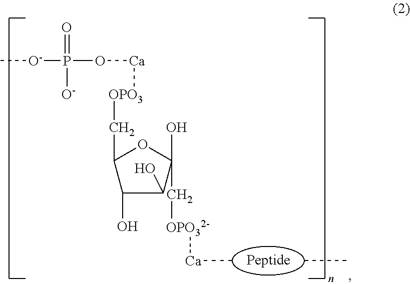

wherein, n is an integer greater than or equal to 1.

In one embodiment, the particle comprises a polymer of succinic acid. In one embodiment, the polymer of succinic acid comprises a structure of formula (3):

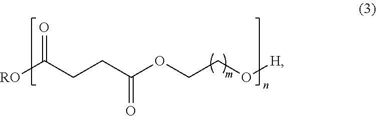

wherein R is a group selected from the group consisting of hydrogen, halogen, alkyl, aryl, heteroaryl, cycloalkyl, heterocyclyl, and combinations thereof; m is an integer from 1 to 30; and n is an integer greater than or equal to 1.

In one embodiment, the polymer of succinic acid comprises the structure,

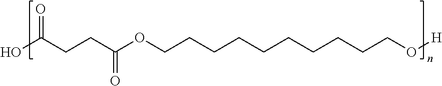

wherein n is an integer greater than or equal to 1.

In one embodiment, the particle comprises a polymer of glutamic acid. In one embodiment, the polymer of glutamic acid comprises a structure of formula (4):

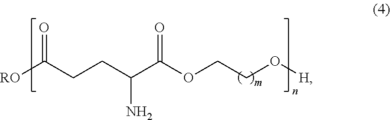

wherein R is a group selected from the group consisting of hydrogen, halogen, alkyl, aryl, heteroaryl, cycloalkyl, heterocyclyl, and combinations thereof; m is an integer from 1 to 30; and n is an integer greater than or equal to 1.

In one embodiment, the polymer of glutamic acid comprises the structure,

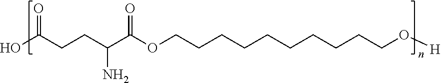

wherein n is an integer greater than or equal to 1.

In one embodiment, the particle comprises a polymer of fructose 6 phosphate. In one embodiment, the polymer of fructose 6 phosphate comprises a structure of formula (5):

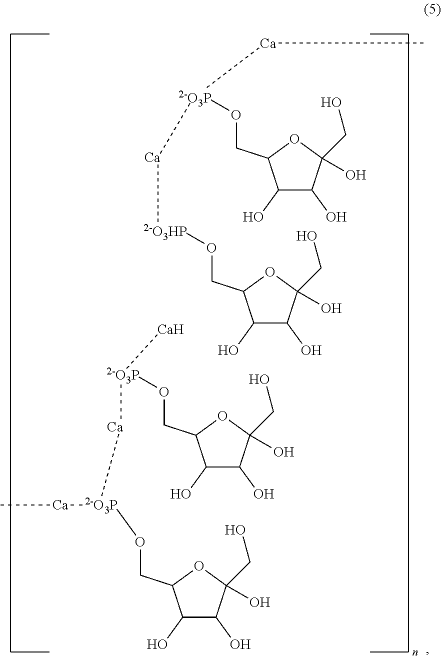

wherein n is an integer greater than or equal to 1.

In one embodiment, the particle comprises a polymer of phosphoenolpyruvic acid. In one embodiment, the polymer of phosphoenolpyruvic acid comprises a structure of formula (6):

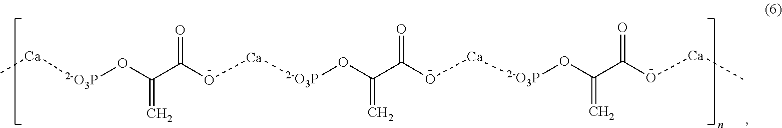

wherein n is an integer greater than or equal to 1.

In one embodiment, the particle provided herein is a particle having any suitable size. For example, in one embodiment, the particle is able to be phagocytosed by a dendritic cell. Thus, in one embodiment, the particle has an average diameter of less than about 15 μm. In one embodiment, the particle has an average diameter of about 5 nm to about 15 μm. In one embodiment, the particle has an average diameter of about 5 nm to about 15 μm. In one embodiment, the particle has an average diameter of about 100 nm to about 10 μm. In one embodiment, the particle has an average diameter of about 100 nm to about 10 μm. In one embodiment, the particle has an average diameter of about 600 nm to about 10 μm. In one embodiment, the particle has an average diameter of about 200 nm to about 300 nm. In one embodiment, the particle has an average diameter of about 1 μm to about 10 μm. In one embodiment, the particle has an average diameter of about 1 μm to about 5 μm. In one embodiment, the particle average has a diameter of about 4 μm.

In one embodiment, the particle provided herein is formed from a polymer of a metabolite provided herein having any suitable molecular weight. In one embodiment, the polymer of a metabolite has a molecular weight of about 1 kDa to about 50 kDa. In one embodiment, the polymer of a metabolite has a molecular weight of about 10 kDa to about 30 kDa. In one embodiment, the polymer of a metabolite has a molecular weight of about 15 kDa to about 25 kDa. In one embodiment, the molecular weight of the polymer of a metabolite is the molecular weight as calculated by GPC.

It will be appreciated by one of ordinary skill in the art that particles suitable for use with the presently disclosed methods can exist in a variety of shapes, including, but not limited to, spheroids, rods, disks, pyramids, cubes, cylinders, nanohelixes, nanosprings, nanorings, rod-shaped particles, arrow-shaped particles, teardrop-shaped particles, tetrapod-shaped particles, prism-shaped particles, and a plurality of other geometric and non-geometric shapes. In some embodiments, the presently disclosed particles have a spherical shape.

Further, in some embodiments, the presently disclosed particles can be surface modified, e.g., by covalently attaching PEG, often referred to as being PEGylated. Such particles can be prepared as disclosed in Lai et al., “Rapid transport of large polymeric nanoparticles in fresh undiluted human mucus,” Proc. Natl. Acad. Sci. U.S.A., 104(5):1482-1487 (2007) and Suh et al., “PEGylation of nanoparticles improves their cytoplasmic transport,” Int. J. Nanomed., 2(4), 735-741 (2007).

### Active Agents

In one embodiment, the particles described herein are combined with an active ingredient, e.g., a drug, medication, or therapeutic agent. Active ingredients include, but are not limited to, any component, compound, or small molecule that can be used to bring about a desired effect, e.g., a therapeutic effect. For example, a desired effect can include the diagnosis, cure, mitigation, treatment, or prevention of a disease or condition.

The active ingredient can be adsorbed, encapsulated, entangled, embedded, incorporated, bound to the surface, or otherwise associated with the particle. As used herein, “combined” encompasses adsorbed, encapsulated, associated, entangled, embedded, incorporated, bound to the surface, or any other means for holding two substances or items together. As provided hereinabove, in some embodiments the presently disclosed particles can include a functional group, e.g., a carboxyl group. Other functional groups include, but are not limited to, a sulfhydryl, hydroxyl, and/or amino group. The functional groups can be available, for example, for drug binding (covalent or electrostatic) or for other desired purposes within the scope of the presently disclosed subject matter.

### Protein and Peptide Active Agents

In one embodiment, particle is combined with a protein or peptide. The peptide of the present disclosure may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer. The peptide may alternatively be made by recombinant means or by cleavage from a longer polypeptide. The composition of a peptide may be confirmed by amino acid analysis or sequencing.

The peptides can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides may include unnatural amino acids formed by post-translational modification or by introducing unnatural amino acids during translation. A variety of approaches are available for introducing unnatural amino acids during protein translation.

A peptide or protein of the disclosure may be phosphorylated using conventional methods such as the method described in Reedijk et al. (The EMBO Journal 11(4):1365, 1992).

The particle may also be combined with cyclic peptides. Cyclization of peptide may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the disclosure, cyclic peptides may comprise a beta-turn in the right position. Beta-turns may be introduced into the peptides of the disclosure by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptide may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis). By way of example, a peptide may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the disclosure conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain a peptide fused to the selected protein or marker protein as described herein. Examples of proteins which may be used to prepare fusion proteins include immunoglobulins, glutathione-S-transferase (GST), hemagglutinin (HA), and truncated myc.

Peptides may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

Peptides and proteins may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one aspect, the particle is combined with an antibody, or antibody fragment, specific for a target. That is, the antibody can inhibit a target to provide a beneficial effect.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain FV molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Small Molecule Active Agents

In various embodiments, the active agent is a small molecule. When the active agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule active agent comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determine the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the disclosure embraces all salts and solvates of the inhibitors depicted here, as well as the non-salt and non-solvate form of the inhibitors, as is well understood by the skilled artisan. In some embodiments, the salts of the inhibitors of the disclosure are pharmaceutically acceptable salts.

Where tautomeric forms may be present for any of the inhibitors described herein, each and every tautomeric form is intended to be included in the present disclosure, even though only one or some of the tautomeric forms may be explicitly depicted. For example, when a 2-hydroxypyridyl moiety is depicted, the corresponding 2-pyridone tautomer is also intended.

The disclosure also includes any or all of the stereochemical forms, including any enantiomeric or diastereomeric forms of the inhibitors described. The recitation of the structure or name herein is intended to embrace all possible stereoisomers of inhibitors depicted. All forms of the inhibitors are also embraced by the disclosure, such as crystalline or non-crystalline forms of the inhibitors. Compositions comprising an inhibitor of the disclosure are also intended, such as a composition of substantially pure inhibitor, including a specific stereochemical form thereof, or a composition comprising mixtures of inhibitors of the disclosure in any ratio, including two or more stereochemical forms, such as in a racemic or non-racemic mixture.

In one embodiment, the small molecule active agent comprises an analog or derivative of an active agent described herein. In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule active agents described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule active agents described herein or can be based on a scaffold of a small molecule active agent described herein, but differing from it in respect to certain components or structural makeup, which may have a similar or opposite action metabolically. An analog or derivative of any of a small molecule inhibitor in accordance with the present disclosure can be used to treat a disease or disorder.

In one embodiment, the small molecule active agents described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Active Agents

In other related aspects, the active agent is an isolated nucleic acid. In certain embodiments, the isolated nucleic acid molecule is one of a DNA molecule or an RNA molecule. In certain embodiments, the isolated nucleic acid molecule is a cDNA, mRNA, or miRNA molecule. In one embodiment, the isolated nucleic acid molecule encodes a therapeutic peptide. In some instances, the active agent is an siRNA, miRNA, or antisense molecule, which inhibits a targeted nucleic acid. In one embodiment, the nucleic acid comprises a promoter/regulatory sequence such that the nucleic acid is preferably capable of directing expression of the nucleic acid. Thus, the disclosure encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In another aspect of the disclosure, a targeted gene or protein, can be inhibited by way of inactivating and/or sequestering the targeted gene or protein. As such, inhibiting the activity of the targeted gene or protein can be accomplished by using a nucleic acid molecule encoding a transdominant negative mutant.

In one embodiment, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present disclosure also includes methods of decreasing levels of PTPN22 using RNAi technology.

In another aspect, the disclosure includes a vector comprising an siRNA or antisense polynucleotide. Preferably, the siRNA or antisense polynucleotide is capable of inhibiting the expression of a target polypeptide. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a nanoparticle of the disclosure. In other embodiments, the selectable marker may be carried on a separate piece of DNA and also be contained within the nanoparticle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the nanoparticle may contain a vector, comprising the nucleotide sequence or the construct to be delivered. The choice of the vector will depend on the host cell in which it is to be subsequently introduced. In a particular embodiment, the vector of the disclosure is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In one embodiment of the disclosure, an antisense nucleic acid sequence, which is expressed by a plasmid vector is used as an active agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In one embodiment of the disclosure, a ribozyme is used as an active agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, Calif.) or they may be genetically expressed from DNA encoding them.

In one embodiment, the active agent may comprise one or more components of a CRISPR-Cas system, where a guide RNA (gRNA) targeted to a gene encoding a target molecule, and a CRISPR-associated (Cas) peptide form a complex to induce mutations within the targeted gene. In one embodiment, the active agent comprises a gRNA or a nucleic acid molecule encoding a gRNA. In one embodiment, the active agents comprise a Cas peptide or a nucleic acid molecule encoding a Cas peptide.

### Compositions

The disclosure provides compositions comprising a particle of the disclosure. In one embodiment, the composition comprises a plurality of particles. The particles can be all nanoparticles, all microparticles, or a combination of nanoparticles and microparticles.

### Pharmaceutical Compositions

The present disclosure also provides pharmaceutical compositions comprising one or more particles of the present disclosure or compositions comprising one or more particles of the present disclosure. The relative amounts of the particles, the pharmaceutically acceptable carrier, and any additional ingredients in a pharmaceutical composition of the disclosure will vary, depending upon the identity, size, and condition of the subject treated and further depending upon the route by which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients. Said compositions may comprise additional medicinal agents, pharmaceutical agents, carriers, buffers, adjuvants, dispersing agents, diluents, and the like depending on the intended use and application.

Examples of suitable pharmaceutical carriers, excipients and/or diluents are well known in the art and include, but are not limited to, a gum, a starch (e.g., corn starch or pregelatinized starch), a sugar (e.g., lactose, mannitol, sucrose, dextrose), a cellulosic material (e.g., microcrystalline cellulose), an acrylate (e.g., polymethacrylate), calcium carbonate, magnesium oxide, talc, or mixtures thereof.

Pharmaceutically acceptable carriers for liquid formulations are aqueous or non-aqueous solutions, suspensions, emulsions or oils, Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, turmeric oil, fish-liver oil, another marine oil, or a lipid from milk or eggs.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well-known conventional methods. Suitable carriers may comprise any material which, when combined with the biologically active compound of the disclosure, retains the biological activity. Preparations for parenteral administration may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles may include sodium chloride solution, Ringer's dextrose, dextrose and sodium chloride, lactated Ringer's, or fixed oils. Intravenous vehicles may include fluid and nutrient replenishes, electrolyte replenishers (such as those based on Ringer's dextrose), and the like. Preservatives and other additives may also be present including, for example, antimicrobials, anti-oxidants, chelating agents, and inert gases and the like, in addition, the pharmaceutical composition of the present disclosure might comprise proteinaceous carriers, e.g., serum albumin or immunoglobulin, preferably of human origin.

Composition comprising particles of the disclosure may be administered alone, or in combination with other drugs and/or agents as pharmaceutical compositions. The composition may contain one or more added materials such as carriers and/or excipients. As used herein, “carriers” and “excipients” generally refer to substantially inert, non-toxic materials that do not deleteriously interact with other components of the composition. These materials may be used to increase the amount of solids in particulate pharmaceutical compositions, such as to form a powder of drug particles. Examples of suitable carriers include water, silicone, gelatin, waxes, and the like.

Examples of normally employed “excipients,” include pharmaceutical grades of mannitol, sorbitol, inositol, dextrose, sucrose, lactose, trehalose, dextran, starch, cellulose, sodium or calcium phosphates, calcium sulfate, citric acid, tartaric acid, glycine, high molecular weight polyethylene glycols (PEG), and the like and combinations thereof. In one embodiment, the excipient may also include a charged lipid and/or detergent in the pharmaceutical compositions. Suitable charged lipids include, without limitation, phosphatidylcholines (lecithin), and the like. Detergents will typically be a nonionic, anionic, cationic or amphoteric surfactant. Examples of suitable surfactants include, for example, Tergitol® and Triton® surfactants (Union Carbide Chemicals and Plastics, Danbury, Conn.), polyoxyethylenesorbitans, for example, TWEEN surfactants (Atlas Chemical Industries, Wilmington, Del.), polyoxyethylene ethers, for example, Brij®, pharmaceutically acceptable fatty acid esters, for example, lauryl sulfate and salts thereof (SDS), and the like. Such materials may be used as stabilizers and/or anti-oxidants. Additionally, they may be used to reduce local irritation at the site of administration.

In at least one embodiment, the composition is formulated in a lyophilized form. In certain embodiments, the lyophilized formulation of the composition allows for maintaining microcarrier structure and achieving remarkably superior long-term stability conditions which might occur during storage or transportation of the particles.

The relative amounts of the particle of the disclosure, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the particles of the disclosure, a pharmaceutical composition of the disclosure may further comprise one or more additional pharmaceutically active agents.

Controlled- or sustained-release formulations of a pharmaceutical composition of the disclosure may be made using conventional technology.

A formulation of a pharmaceutical composition of the disclosure suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the disclosure which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions comprising the particles of the disclosure in aqueous or oily solvents may be prepared in substantially the same manner as liquid suspensions, the primary difference being that the active ingredient is dissolved, rather than suspended in the solvent. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the disclosure formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface-active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the disclosure.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the disclosure may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers and may further comprise one or more of the additional ingredients described herein.

A pharmaceutical composition of the disclosure may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more of the other additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Genaro, ed., 1985, Remington's Pharmaceutical Sciences, Mack Publishing Co., Easton, Pa., which is incorporated herein by reference.

### Scaffolds and Substrates

In one embodiment, disclosure provides a scaffold or substrate composition comprising a particle composition of the disclosure. For example, in one embodiment, a composition comprising a particle of the disclosure is within a scaffold. In another embodiment a composition comprising a particle of the disclosure is applied to the surface of a scaffold. The scaffold of the disclosure may be of any type known in the art. Non-limiting examples of such a scaffold includes a, hydrogel, electrospun scaffold, foam, mesh, sheet, patch, sponge, stents, and other biomaterial implants.

### Hydrogels

In one embodiment, the present disclosure provides a hydrogel comprising a composition comprising a particle of the disclosure. Hydrogels can generally absorb a great deal of fluid and, at equilibrium, typically are composed of 60-90% fluid and only 10-30% polymer. In one embodiment, the water content of hydrogel is about 70-80%. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility may be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27).

In one embodiment, the composition comprises a hydrogel comprising a particle of the disclosure. A hydrogel may comprise one or more other biopolymer or synthetic polymer. The hydrogels may be prepared by crosslinking hydrophilic biopolymers or synthetic polymers. Examples of the hydrogels formed from physical or chemical crosslinking of hydrophilic biopolymers, include but are not limited to, hyaluronans, chitosans, alginates, collagen, dextran, pectin, carrageenan, polylysine, gelatin or agarose. (see.: W. E. Hennink and C. F. van Nostrum, 2002, Adv. Drug Del. Rev. 54, 13-36 and A. S. Hoffman, 2002, Adv. Drug Del. Rev. 43, 3-12). These materials consist of high-molecular weight backbone chains made of linear or branched polysaccharides or polypeptides. Examples of hydrogels based on chemical or physical crosslinking synthetic polymers include but are not limited to (meth)acrylate-oligolactide-PEO-oligolactide-(meth)acrylate, poly(ethylene glycol) (PEO), poly(propylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), poly(ethylene glycol) diacrylate (PEGDA), etc. (see A. S Hoffman, 2002 Adv. Drug Del. Rev, 43, 3-12).

In certain embodiments, the hydrogel is modified to comprise one or more therapeutic agents. Hydrogels may be modified with functional groups for covalently attaching a variety of compounds. In one embodiment, compounds, such as therapeutic agents, may be incorporated into the hydrogel matrix.

Additional therapeutic agents which may be incorporated into the hydrogel scaffold include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents.

In certain embodiments, one or more multifunctional cross-linking agents known in the art may be utilized as reactive moieties that covalently link biopolymers or synthetic polymers.

### Electrospun Scaffolds

In one embodiment, one or more particles of the disclosure may be incorporated into nanofibrous biocompatible electrospun matrices. In some embodiments, particles may be blended with a synthetic polymer, such as poly(ethylene oxide) (PEO) to produce a tissue engineering scaffold.

The scaffolds of the disclosure may be produced in a variety of ways. In an exemplary embodiment, the scaffold may be produced by electrospinning. Electrospinning is an atomization process of a conducting fluid which exploits the interactions between an electrostatic field and the conducting fluid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material may be collected as an interconnected web containing relatively fine, i.e. small diameter, fibers. The resulting films (or membranes) from these small diameter fibers have very large surface area to volume ratios and small pore sizes. A detailed description of electrospinning apparatus is provided in Zong, et al., 2002 Polymer 43: 4403-4412; Rosen et al., 1990 Ann Plast Surg 25: 375-87; Kim, K., Biomaterials 2003, 24: 4977-85; Zong, X., 2005 Biomaterials 26: 5330-8. After electrospinning, extrusion and molding may be utilized to further fashion the polymers. To modulate fiber organization into aligned fibrous polymer scaffolds, the use of patterned electrodes, wire drum collectors, or post-processing methods such as uniaxial stretching has been successful. Zong, X., 2005 Biomaterials 26: 5330-8; Katta, P., 2004 Nano Lett 4: 2215-2218; Li, D., 2005 Nano Lett 5: 913-6.

The disclosure also includes combinations of natural materials, combinations of synthetic materials, and combinations of both natural and synthetic materials. For example, the particles may be combined with natural materials, synthetic materials, or both natural and synthetic materials to produce the scaffolds of the disclosure. Examples of combinations include, but are not limited to: blends of different types of collagen (e.g. Type I with Type II, Type I with Type III, Type II with Type III, etc.); blends of one or more types of collagen with fibrinogen, thrombin, elastin, PGA, PLA, and polydioxanone; and blends of fibrinogen with one or more types of collagen, thrombin, elastin, PGA, PLA, and polydioxanone.

In embodiments in which the matrix contains substances that are to be released from the matrix, incorporating electroprocessed synthetic components, such as biocompatible substances, can modulate the release of substances from an electroprocessed composition. For example, layered or laminate structures may be used to control the substance release profile. Unlayered structures may also be used, in which case the release is controlled by the relative stability of each component of the construct. For example, layered structures composed of alternating electroprocessed materials are prepared by sequentially electroprocessing different materials onto a target. The outer layers are, for example, tailored to dissolve faster or slower than the inner layers. Multiple agents may be delivered by this method, optionally at different release rates. Layers may be tailored to provide a complex, multi-kinetic release profile of a single agent over time. Using combinations of the foregoing provides for release of multiple substances released, each with its own profile. Complex profiles are possible.

### Forming Matrices or Scaffolds

A biocompatible scaffold may be shaped using methods such as, for example, solvent casting, compression molding, filament drawing, meshing, leaching, weaving, foaming, electrospinning and coating. In solvent casting, a solution of one or more proteins in an appropriate solvent, is cast as a branching pattern relief structure. After solvent evaporation, a thin film is obtained. In compression molding, a polymer is pressed at pressures up to 30,000 pounds per square inch into an appropriate pattern. Filament drawing involves drawing from the molten polymer and meshing involves forming a mesh by compressing fibers into a felt-like material. In leaching, a solution containing two materials is spread into a shape close to the final form of the artificial organ. Next a solvent is used to dissolve away one of the components, resulting in pore formation. (See U.S. Pat. No. 5,514,378 to Mikos).

The scaffold may be shaped into any number of desirable configurations to satisfy any number of overall system, geometry or space restrictions. For example, in the use of the scaffold for bladder, urethra, valve, or blood vessel reconstruction, the matrix or scaffold may be shaped to conform to the dimensions and shapes of the whole or a part of the tissue. The scaffold may be shaped in different sizes and shapes to conform to the organs of differently sized patients. The matrix or scaffold may also be shaped in other fashions to accommodate the special needs of the patient.

### Solid Supports

In one embodiment, one or more particles of the disclosure may be incorporated into a solid support. The solid support can be any solid support known to a person of skill in the art for its use in bandages, wound healing, wound dressing, and/or wound packaging for initial and/or emergency treatment. In one embodiment, the solid support comprises a natural fiber. Exemplary natural fibers are described elsewhere herein. In one embodiment, the solid support comprises cotton. In one embodiment, the solid support is cotton. In one embodiment, the cotton is a cotton ball. In one embodiment, the solid support comprises chitin. In one embodiment, the solid support is chitin. In one embodiment, the solid support comprises silica

### Methods of Forming Polymers and Particles

In one embodiment, the disclosure provides a method for forming a metabolite-based polymer. In one embodiment, the method provides a polymer of a metabolite comprising at least one carboxylic group. In one embodiment, the method provides a polymer of a metabolite comprising at least two carboxylic groups.

In one embodiment, the method comprises: forming a mixture of the metabolite comprising a carboxylic group and a diol compound of formula (A):

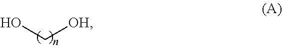

wherein n is an integer from 2 to 30. In one embodiment, the method comprises forming a mixture of the metabolite and forming a mixture of the metabolite comprising a carboxylic group and 1,10-decanediol. In one embodiment, the method comprises forming a mixture of the metabolite and forming a mixture of the metabolite comprising a carboxylic group and 1,4 butanediol. In one embodiment, the method comprises forming a mixture of the metabolite and forming a mixture of the metabolite comprising a carboxylic group and 1,6 hexanediol. In one embodiment, the method comprises forming a mixture of the metabolite and forming a mixture of the metabolite comprising a carboxylic group and 1,8 octanediol.

In one embodiment, the method further comprises heating the mixture at about 100° C. to about 150° C. In one embodiment, the method further comprises heating the mixture at about 130° C.

In one embodiment, the method is a method of forming a aKG based polymer. In one embodiment, the method comprises forming a mixture of aKG and a diol compound of formula (A):

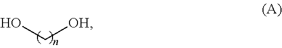

wherein n is an integer from 2 to 30. In one embodiment n is an integer from 4 to 10. In one embodiment n is 4, 6, 8 or 10. In one embodiment, the method comprises forming a mixture of the metabolite and forming a mixture of the metabolite comprising a carboxylic group and 1,10-decanediol. In one embodiment, the method further comprises heating the mixture at about 35° C. to about 200° C. In one embodiment, the method further comprises heating the mixture at about 100° C. to about 150° C. In one embodiment, the method further comprises heating the mixture at about 130° C. In one embodiment, the mixture is heated for about 30 minutes to about 72 hours. In one embodiment, the mixture is heated for about 24 hours to about 72 hours. In one embodiment, the mixture is heated for about 48 hours. In one embodiment, the mixture further comprises a catalyst. In one embodiment, the catalyst is Sn. In one embodiment, the catalyst is for generating an ester bond.

In one embodiment, the disclosure provides a method for producing a particle comprising a metabolite-based polymer, wherein the metabolite comprises at least one carboxylic group. In one embodiment, forming a particle of the polymer comprises a water-oil emulsion. In one embodiment, forming a particle of the polymer comprises a water-oil-water emulsion.

In one embodiment, the disclosure provides a method for producing active metabolite-based polymeric particles. For example, in one embodiment, the disclosure provides a method for producing a particle of a metabolite having a phosphate group. In one embodiment, the method comprises mixing calcium, the metabolite comprising a phosphate group, and a nucleation agent. In one embodiment, the nucleation agent is a negatively charged polymer. In one embodiment, the nucleation agent is as ovalbumin, or poly(sodium 4-styrenesulfonate). In one embodiment, the 4-aminophenyl phosphonic acid is conjugated to the metabolite.

### Methods of Use

In one embodiment, the disclosure provides a method of modulating the intracellular metabolite profile of a dendritic cell. In one embodiment, the method comprises administering a particle comprising a polymer of a metabolite to the subject. In one embodiment, the particle delivers the metabolite to the subject and modulates the intracellular metabolite profile of one or more dendritic cells in the subject.

In one embodiment, the method modulates the glutamate pathway of the dendritic cell. In one embodiment, the method increases one or more of L-glutamate, 4-aminobutanoate and asparagine in the cell. In one embodiment, the method modulates the arginine pathway of the cell. In one embodiment, the method increases one or more of aspartate, acetyl-ornithine and L-citrulline in the cell. In one embodiment, the method increases kynurenine in the cell.

In one embodiment, the disclosure provides a method of decreasing glycolysis in a dendritic cell. In one embodiment, the method comprises administering a particle comprising a polymer of a metabolite to the subject. In one embodiment, the particle delivers the metabolite to the subject and decreasing glycolysis in one or more dendritic cells in the subject.

In one embodiment, the disclosure provides a method of modulating cytokine production in a dendritic cell. In one embodiment, the method comprises administering a particle comprising a polymer of a metabolite to the subject. In one embodiment, the particle delivers the metabolite to the subject and modulates cytokine production in one or more dendritic cells in the subject.

In one embodiment, the disclosure provides a method of decreasing pro-inflammatory T cell responses in a subject. In one embodiment, the method comprises administering a particle comprising a polymer of a metabolite to the subject. In one embodiment, the method decreases Th1, Th2, Th17 and Treg population in the subject.

In one embodiment, the disclosure provides a method of facilitating wound healing in a subject. In one embodiment, the method comprises administering a particle comprising a polymer of a metabolite to the subject.

In one embodiment, the disclosure provides a method of modulating immune response in a subject. In one embodiment, the method comprises administering a particle comprising a polymer of a metabolite to the subject. In one embodiment, the particle delivers the metabolite to the subject and activates metabolic pathways. In one embodiment, the particle delivers the metabolite to the subject and rescues immune cells against metabolic exhaustion.

The present disclosure also provides a method of treating or preventing a disease or disorder in a subject. In one embodiment, the method comprises administering an effective amount of a composition comprising the nanoparticle of the disclosure to a subject in need thereof. In one embodiment, the polymeric particle comprises at least one therapeutic agent to treat the patient's disease or disorder. In one embodiment, the disease or disorder is associated with increased immune activation.

Exemplary diseases or disorders that can be treated using the compositions and methods of the disclosure include, but are not limited to, inflammatory diseases and disorders, and autoimmune diseases and disorders. Exemplary diseases that can be treated using the compositions and methods of the disclosure include, but are not limited to, rheumatoid arthritis/seronegative arthropathies, osteoarthritis, inflammatory bowel disease, systemic lupus erythematosis, iridoeyelitis/uveitistoptic neuritis, idiopathic pulmonary fibrosis, systemic vasculitis/Wegener's granulomatosis, sarcoidosis, including, but not limited to, rheumatoid arthritis/seronegative arthropathies, osteoarthritis, inflammatory bowel disease, systemic lupus erythematosis, iridoeyelitis/uveitistoptic neuritis, idiopathic pulmonary fibrosis, systemic vasculitis/Wegener's granulomatosis, sarcoidosis, myocarditis, postmyocardial infarction syndrome, postpericardiotomy syndrome, subacute bacterial endocarditis (SBE), anti-glomerular basement membrane nephritis, interstitial cystitis, lupus nephritis, autoimmune hepatitis, primary biliary cholangitis (PBC), primary sclerosing cholangitis, antisynthetase syndrome, alopecia areata, autoimmune angioedema, autoimmune progesterone dermatitis, autoimmune urticaria, bullous pemphigoid, cicatricial pemphigoid, dermatitis herpetiformis, discoid lupus erythematosus, epidermolysis bullosa acquisita, erythema nodosum, gestational pemphigoid, hidradenitis suppurativa, lichen planus, lichen sclerosus, linear IgA disease (LAD), morphea, pemphigus vulgaris, pityriasis lichenoides et varioliformis acuta, Mucha-Habermann disease, psoriasis, systemic scleroderma, vitiligo, Addison's disease, autoimmune polyendocrine syndrome (APS) type 1, autoimmune polyendocrine syndrome (APS) type 2, autoimmune polyendocrine syndrome (APS) type 3, autoimmune pancreatitis (AIP), diabetes mellitus type 1, autoimmune thyroiditis, Ord's thyroiditis, Graves' disease, autoimmune oophoritis, endometriosis, autoimmune orchitis, Sjogren's syndrome, autoimmune enteropathy, Coeliac disease, Crohn's disease, microscopic colitis, ulcerative colitis, antiphospholipid syndrome (APS, APLS), aplastic anemia, autoimmune hemolytic anemia, autoimmune lymphoproliferative syndrome, autoimmune neutropenia, autoimmune thrombocytopenic purpura, cold agglutinin disease, essential mixed cryoglobulinemia, Evans syndrome, pernicious anemia, pure red cell aplasia, thrombocytopenia, adiposis dolorosa, adult-onset Still's disease, ankylosing spondylitis, CREST syndrome, drug-induced lupus, enthesitis-related arthritis, eosinophilic fasciitis Felty syndrome, IgG4-related disease, juvenile arthritis, Lyme disease (chronic), mixed connective tissue disease (MCTD), palindromic rheumatism, Parry Romberg syndrome, Parsonage-Turner syndrome, psoriatic arthritis, reactive arthritis, relapsing polychondritis, retroperitoneal fibrosis, rheumatic fever, Schnitzler syndrome, undifferentiated connective tissue disease (UCTD), dermatomyositis, fibromyalgia, inclusion body myositis, myositis, myasthenia gravis, neuromyotonia, paraneoplastic cerebellar degeneration, polymyositis, acute disseminated encephalomyelitis (ADEM), acute motor axonal neuropathy, anti-N-methyl-D-aspartate (Anti-NMDA) receptor encephalitis, balo concentric sclerosis, Bickerstaff's encephalitis, chronic inflammatory demyelinating polyneuropathy, Guillain-Barré syndrome, Hashimoto's encephalopathy, idiopathic inflammatory demyelinating diseases, Lambert-Eaton myasthenic syndrome, multiple sclerosis, pattern II, Oshtoran Syndrome, pediatric autoimmune neuropsychiatric disorder associated with streptococcus (PANDAS), progressive inflammatory neuropathy, restless leg syndrome, stiff person syndrome, sydenham chorea, transverse myelitis, autoimmune retinopathy, autoimmune uveitis, Cogan syndrome, Graves ophthalmopathy, intermediate uveitis, ligneous conjunctivitis, Mooren's ulcer, neuromyelitis optica, opsoclonus myoclonus syndrome, optic neuritis, scleritis, Susac's syndrome, sympathetic ophthalmia, Tolosa-Hunt syndrome, autoimmune inner ear disease (AIED), Ménière's disease, Behçet's disease, eosinophilic granulomatosis with polyangiitis (EGPA), giant cell arteritis, granulomatosis with polyangiitis (GPA), IgA vasculitis (IgAV), Kawasaki's disease, leukocytoclastic vasculitis, lupus vasculitis, rheumatoid vasculitis, microscopic polyangiitis (MPA), polyarteritis nodosa (PAN), polymyalgia rheumatic, urticarial vasculitis, vasculitis, and primary immune deficiency. In one embodiment, the disease is rheumatoid arthritis.

In one embodiment, the disclosure provides a method of reducing or preventing organ transplant rejection. In one embodiment, the method comprises administering an effective amount of a composition comprising the nanoparticle of the disclosure to a subject in need thereof. In one embodiment, the method confers improved or superior retention of organ transplants.

In one embodiment, the disclosure provides methods for administering a biomaterial to a subject. In one embodiment, the method comprises administering a biomaterial to the subject, wherein the biomaterial is coated with a composition of the disclosure. In one embodiment, the biomaterial is a stent.

In various embodiments, the composition comprising the metabolite-based polymer of the disclosure is administered to a subject in need in a wide variety of ways. In various embodiments, the polymeric particle, or pharmaceutical composition comprising the polymeric particle, of the disclosure is administered orally, intraoperatively, intravenously, intravascularly, intramuscularly, subcutaneously, intracerebrally, intraperitoneally, by soft tissue injection, by surgical placement, by arthroscopic placement, and by percutaneous insertion, e.g., direct injection, cannulation or catheterization. Any administration may be a single administration of a composition of disclosure or multiple administrations. Administrations may be to single site or to more than one site in the subject being treated. Multiple administrations may occur essentially at the same time or separated in time.

Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions of the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present disclosure to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present disclosure are administered to a patient by intradermal or subcutaneous injection. In another embodiment, the compositions of the present disclosure are preferably administered by i.v. injection.

The composition comprising the polymeric particle described herein can be incorporated into any formulation known in the art. For example, the polymeric particle may be incorporated into formulations suitable for oral, parenteral, intravenous, subcutaneous, percutaneous, topical, buccal, or another route of administration. Suitable compositions include, but are not limited to, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

In the method of treatment, the administration of the composition of the disclosure may be for either “prophylactic” or “therapeutic” purpose. When provided prophylactically, the composition of the present disclosure is provided in advance of any sign or symptom, although in particular embodiments the disclosure is provided following the onset of at least one sign or symptom to prevent further signs or symptoms from developing or to prevent present signs or symptoms from becoming more severe. The prophylactic administration of the composition serves to prevent or ameliorate subsequent signs or symptoms. When provided therapeutically, the pharmaceutical composition is provided at or after the onset of at least one sign or symptom. Thus, the present disclosure may be provided either prior to the anticipated exposure to a disease-causing agent or disease state or after the initiation of the disease or disorder.

### EXPERIMENTAL EXAMPLES

The invention is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, are not to be construed as limiting in any way the remainder of the disclosure.

### Example 1: Metabolite Delivery for Modulating Metabolic Pathways of Cells

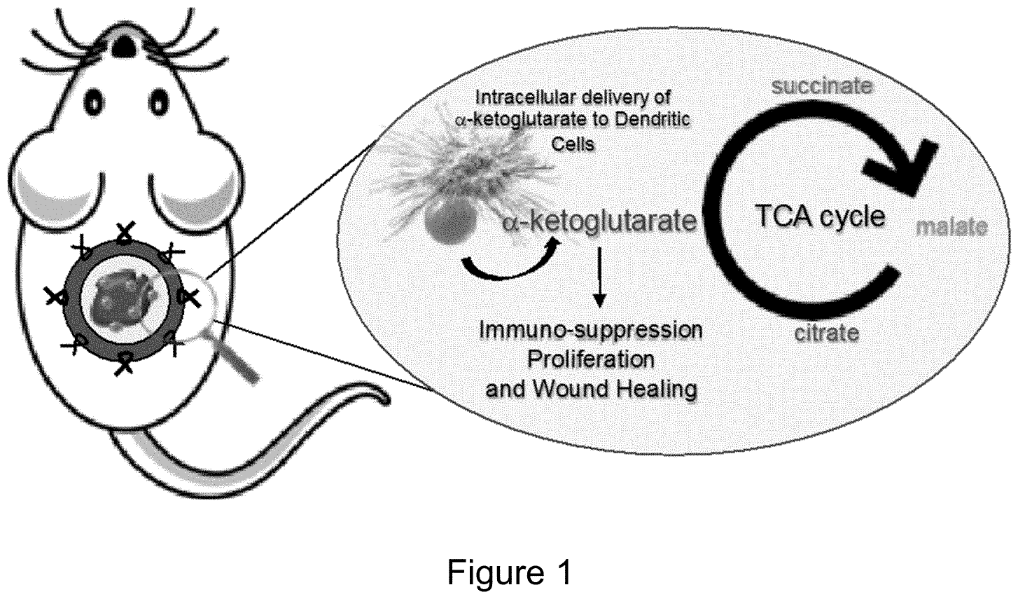

The data presented herein demonstrates novel nanoparticles generated from polymer of fructose, 1,6,biphosphate (pF16BP-glycolysis accelerator), and poly(succinate) (poly(succinate)-tricarboxylic acid cycle (PS-TCA) accelerator) were able to rescue the proliferation/activation of T-cells in mixed-lymphocyte-reaction from glycolysis/glutaminolysis inhibition in vitro. Moreover, these particles were able to activate dendritic cells (DCs) differentially (FIG. 1).

### Metabolites with Phosphate Groups

Traditionally, calcium phosphate particles are generated by adding specific amounts of calcium and phosphate groups to plasmid DNA. Here, the phosphate groups of different metabolites, which are used by cells for generating energy, are used to generate calcium phosphate particles. Calcium was added to these phosphate groups to generate the particles in the presence of a nucleation agent. The nucleation was generated by addition of a negatively charged polymer such as ovalbumin (protein), Poly(sodium 4-styrenesulfonate) among others.

Moreover, in order to increase yield in case of low negatively charged polymers (e.g. ovalbumin), 4-aminophenyl phosphonic acid was conjugated to the proteins using (1-Ethyl-3-(3-dimethylaminopropyl)-carbodiimide) and sulfo-N-hydroxysuccinimide chemistry. The presence of phosphate groups provides a nucleation site for the growth of the nanoparticles on the proteins.

The particles were generated using different metabolites with phosphate groups, thereby showing the versatility of the process. The metabolites used to generate the particles were Fructose, 1,6 biphosphate, fructose 6 phosphate, and phosphoenolpyruvic acid.

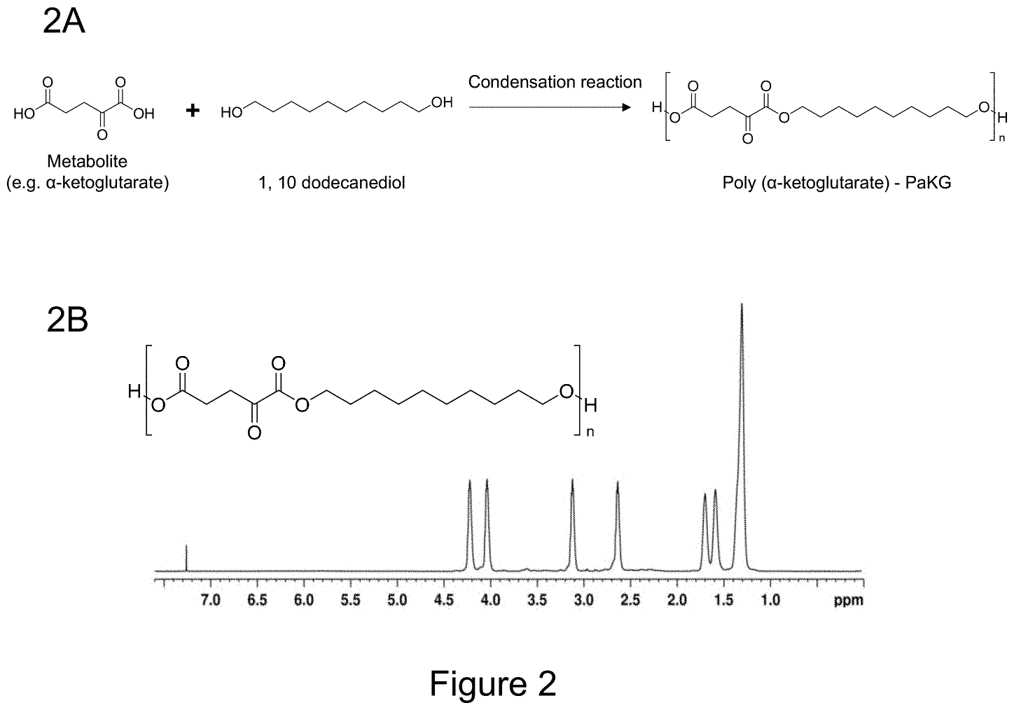

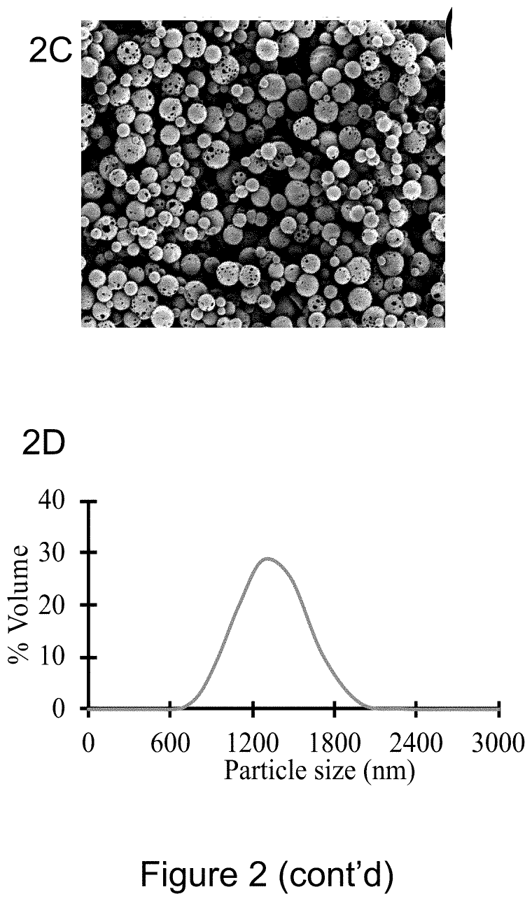

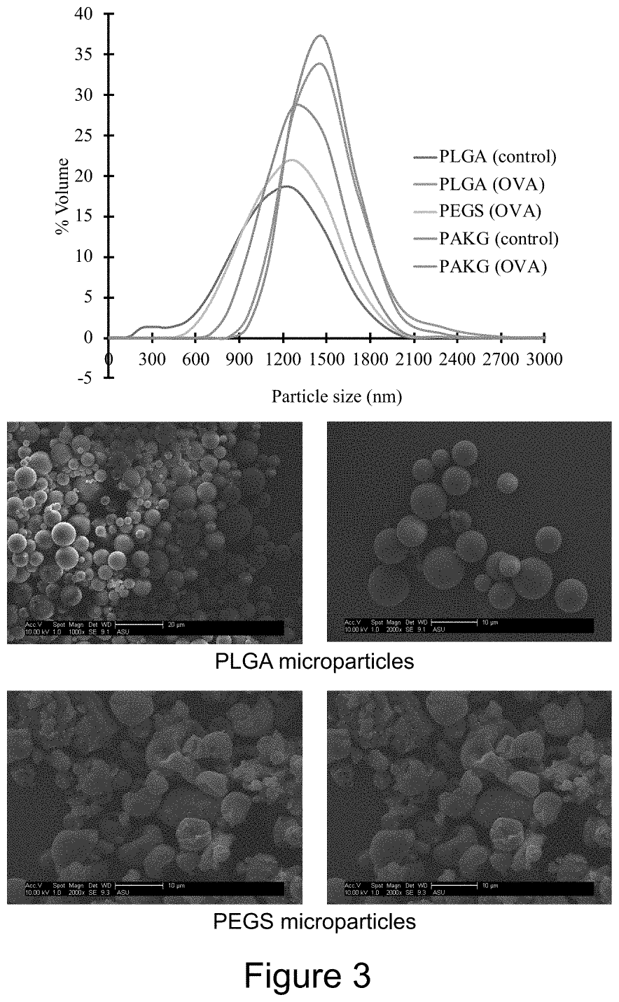

Dynamic light scattering was utilized to measure the size of the particles. The size of the particles was observed to be 100-300 nm (FIG. 2).

### Metabolites with Two Carboxylic Acid Groups

In order to generate polymers that can release metabolites in a sustained fashion, metabolites from Kreb's cycle and 1,12 dodecanediol were utilized. Specifically, polymers were generated by using metabolites—alpha-ketoglutarate or succinic acid or glutamic acid. As an example of the synthesis—alpha-ketoglutarate was added to the synthesis pot with 1,12 dodecanediol and heated to 130 degrees Celsius for 48 hours to generate the polymers. The polymers were then purified by washing in diethyl ether several times (>3 times with >10 times the volume). The polymers were then dried for 48 hours to generate a purified polymer.

### Particle Synthesis

In order to generate microparticles with these novel polymers water-oil emulsion (for encapsulating hydrophobic molecules) or water-oil-water emulsion (for encapsulating proteins) methods were utilized. Briefly, dicholoromethane (DCM) as an oil was utilized to dissolve the polymers at 50 mg/mL concentration. If hydrophobic molecules, such as CB-839 (glutaminase inhibitor), PFK15 (glycolysis inhibitor) or fluorescent molecules (rhodamine 6G, fluorescein) were to be encapsulated then they were directly added to the dichloromethane directly. This solution was then added to 2% polyvinylalcohol solution made in double deionized water and homogenized at different speeds to generate a stable emulsion. DCM was evaporated and the solid particles were obtained.

In order to encapsulate hydrophilic molecules (e.g. ovalbumin proteins, bovine collagen type II etc.), these molecules were added to the primary emulsion and sonicated using a sonicated probe. This primary emulsion as then added to the 2% polyvinylalcohol solution made in double deionized water and homogenized at different speeds to generate a stable emulsion. DCM was evaporated and the solid particles were obtained.

The size of the particles thus generated was obtained to be 1-5 micrometer using Dynamic light scattering. Scanning electron microscopy was also utilized to observe the particle morphology.

### Release Kinetics

The polymers thus generated were incubated in acetate buffer pH=5 or phosphate buffered saline, pH=7.4, and the release of the metabolites was studied using 1H NMR for 30 days. It was observed that the polymer particles were able to release Kreb's cycle metabolites for 30 days.

### Modulation of Immune Responses

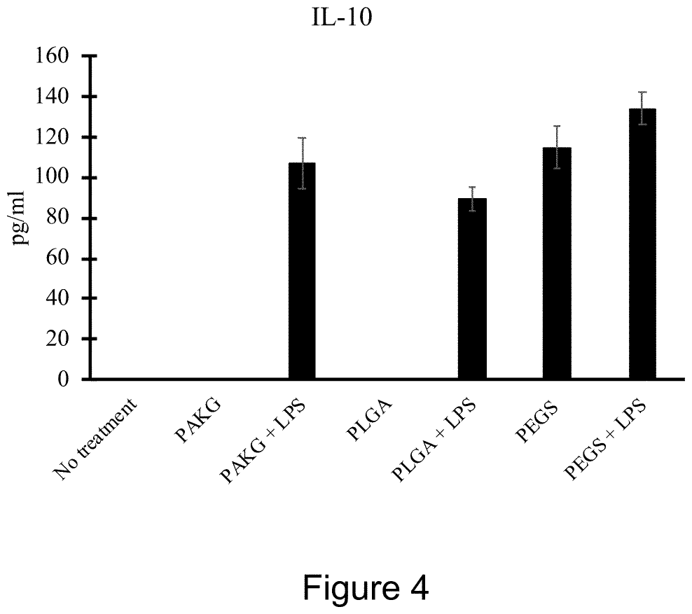

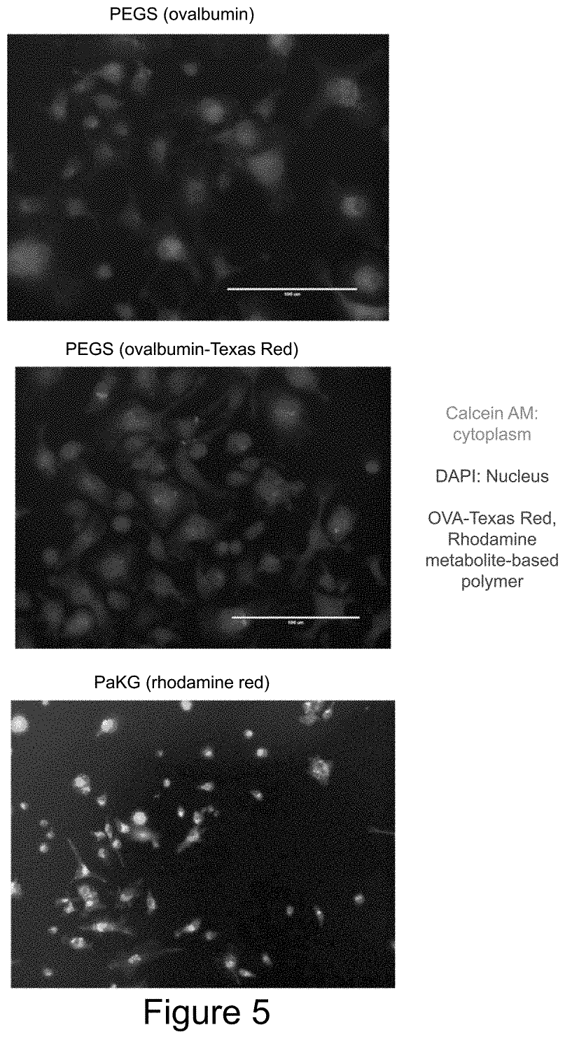

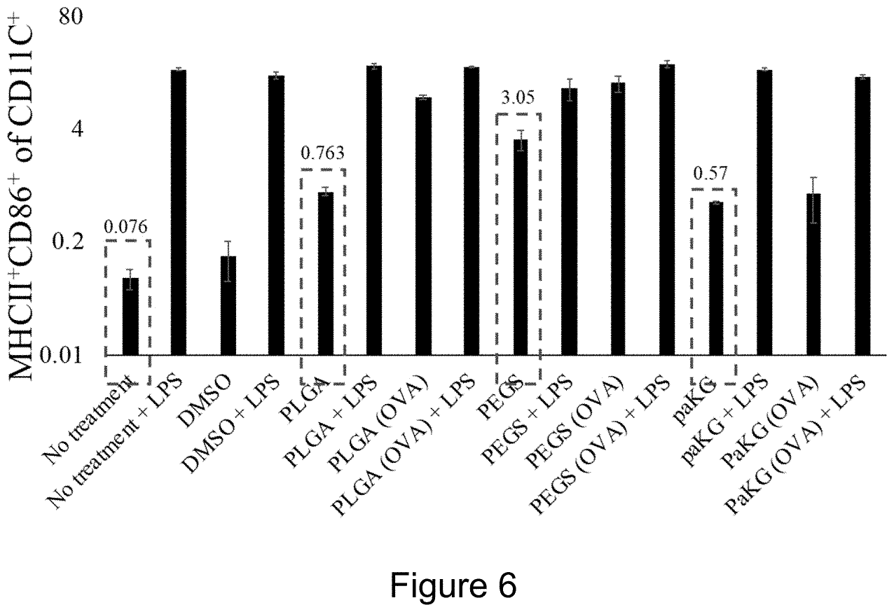

The particles thus generated were added to the cell culture. Dendritic cells (DCs), a specialized immune cell, were obtained from the bone marrow of mice after a 10 day culture. These cells were then treated with different amounts of these particles. It was observed that the particles depending on the type of metabolites, differentially upregulated the activation of dendritic cells. The activation as observed by staining the surface marker, CD11c, CD86, and MHC-II of DCs. Moreover, ELISA was utilized to observe the IL-12p70 (pro-inflammatory) and IL-10 (anti-inflammatory—FIG. 4) production by dendritic cells.

It was observed that poly succinate increased the activation of the DCs, followed by PLGA (control) and lowest activation was observed in case of poly alpha-ketoglutarate. Moreover, a 72 hour syngeneic mixed lymphocyte reaction was performed between DCs obtained from bone marrow and CD3+ T-cells obtained (magnetic separation Mojo cell separation protocol) from spleen of C57BL6/j. Particles were added to these cultures and the amount of activation in T-cells was observed using flow cytometry. Upon phagocytosis of these microparticles by DCs, T-cell suppression or activation was observed in vitro by staining for surface markers (CD4, CD8, CD25) and intracellular markers (Tbet, RORyT, FOXp3, Ki67, and GATA3).

### Example 2: Alpha-Ketoglutaric Acid-Based Polymers Induce Immune Suppression by Modulating Metabolism of Dendritic Cells

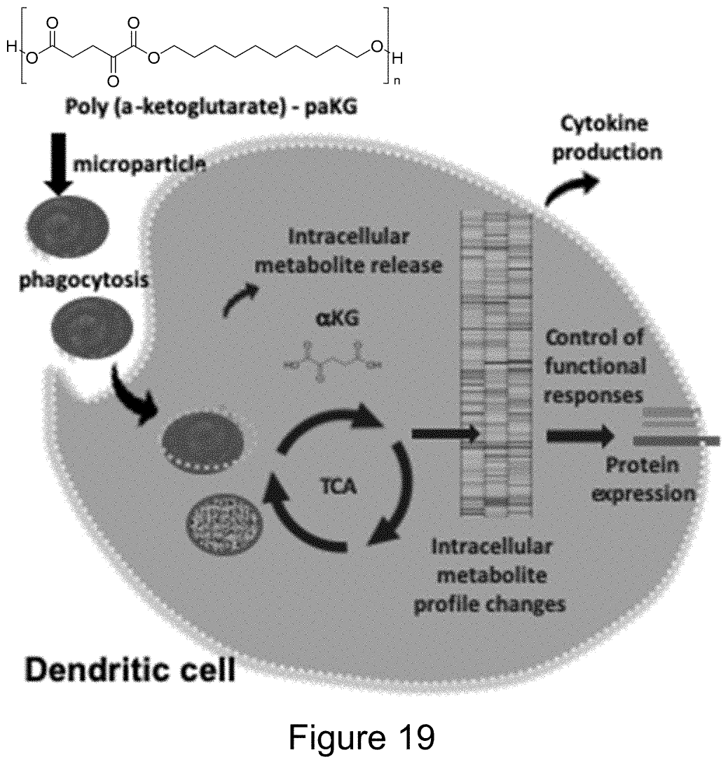

The data presented herein demonstrates the synthesis of alpha-ketoglutarate (aKG)-based polymeric-microparticles (termed PaKG MPs) to provide sustained release of aKG and promote an immunosuppressive cellular phenotype (FIG. 19). Notably, after phagocytosis by dendritic cells (DCs), PaKG MPs modulated the intracellular metabolic-profile/pathways, and decreased glycolysis and mitochondrial respiration in vitro. These metabolic changes resulted in modulation of MHC-II, CD86, IL-12p70, IL-10 and TNF-alpha expression in DCs, and altered the frequency of regulatory T cells (Tregs), and T-helper type-1/2/17 cells in vitro. Importantly, in vivo, PaKG MPs increased Th2 frequency in draining lymph nodes of cutaneous wounds in mice, and accelerated wound closure compared to soluble aKG and saline controls. This unique strategy of intracellular delivery of key-metabolites provide a paradigm-shift in the immunometabolism field-based immunotherapy with applications in different diseases associated with immune disorders.

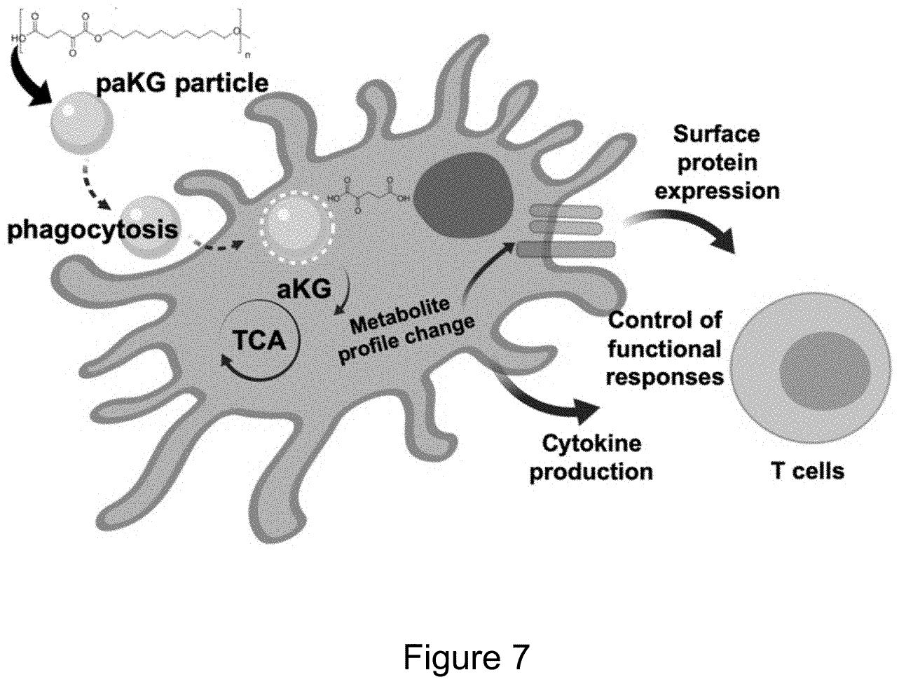

Further, these data demonstrate that sustained delivery of aKG (a Kreb's cycle metabolite, and involved in immunosuppression (Liu et al., Nat. Immunol. 18(9):985-994, 2017)) after a one-time application can not only lead to modulation of the dendritic cell metabolism, but also may play a role in the proliferation and repair phase of wound healing and thus lead to accelerated wound healing responses (FIG. 7). Interestingly, aKG-based polymers could modulate the intracellular metabolite profile of DCs and down-regulate a set of metabolites known to traditionally activate DCs. Importantly, aKG-based polymers modulate the DC phenotype and subsequent adaptive immune responses in the form of T cells in vitro and in vivo.

### Central-Carbon aKG Metabolite-Based Microparticles (MPs) Release aKG in a Sustained Manner

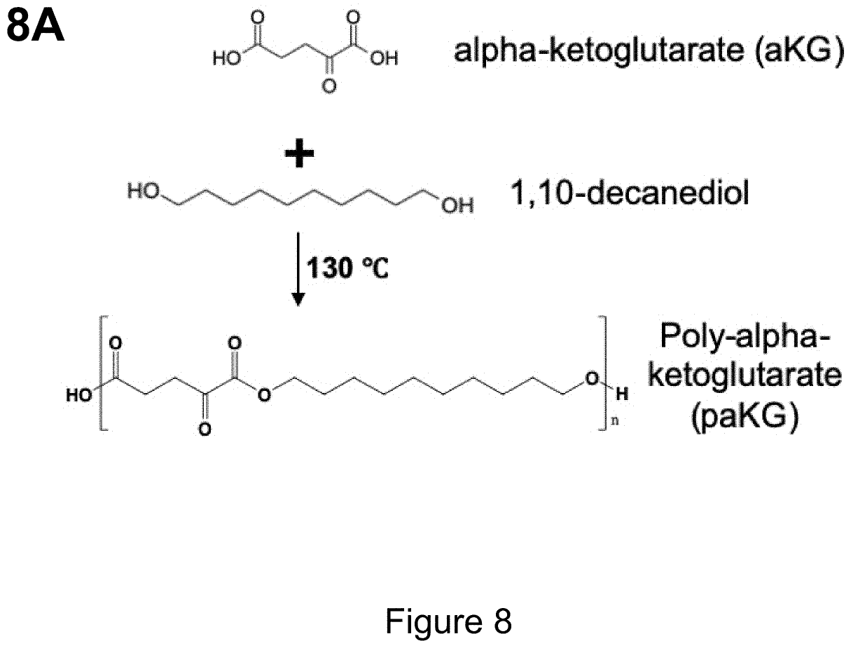

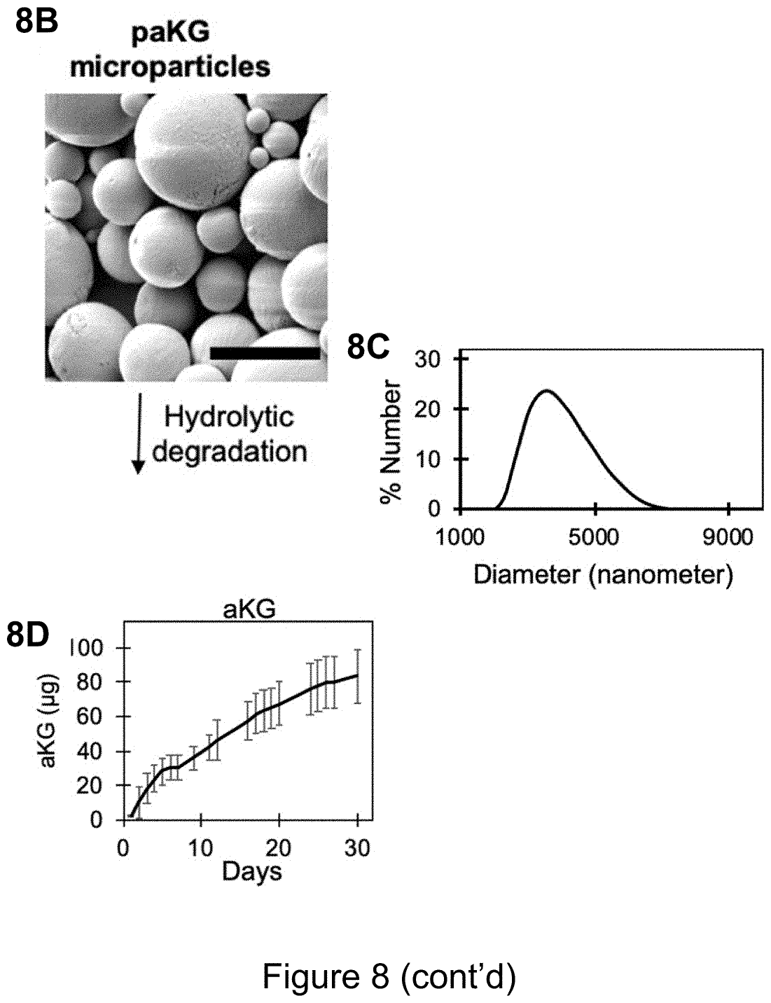

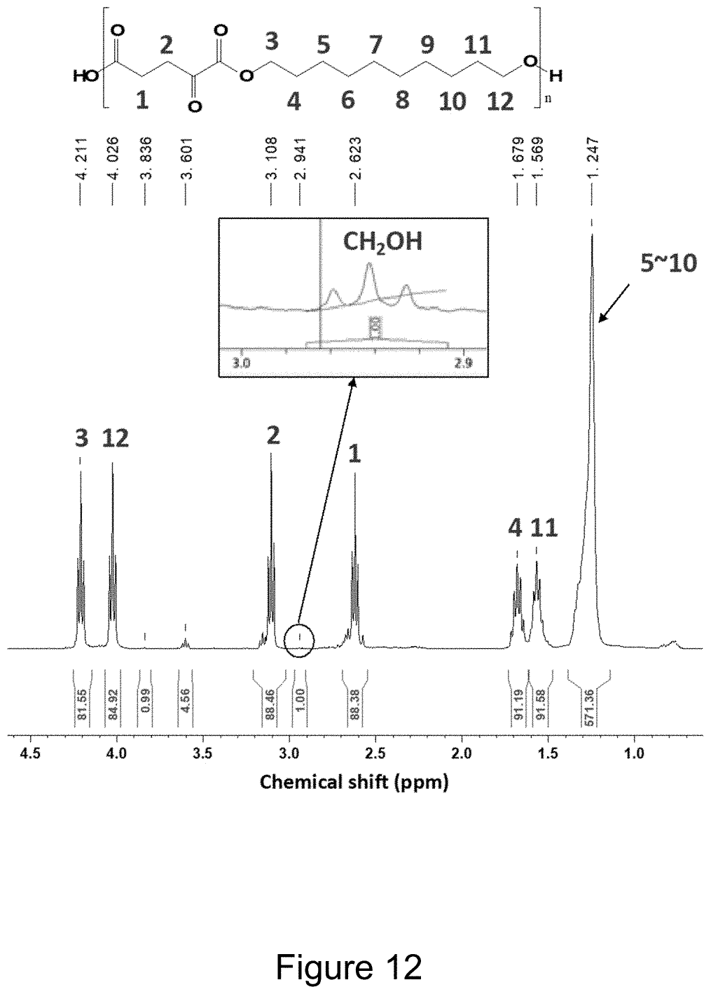

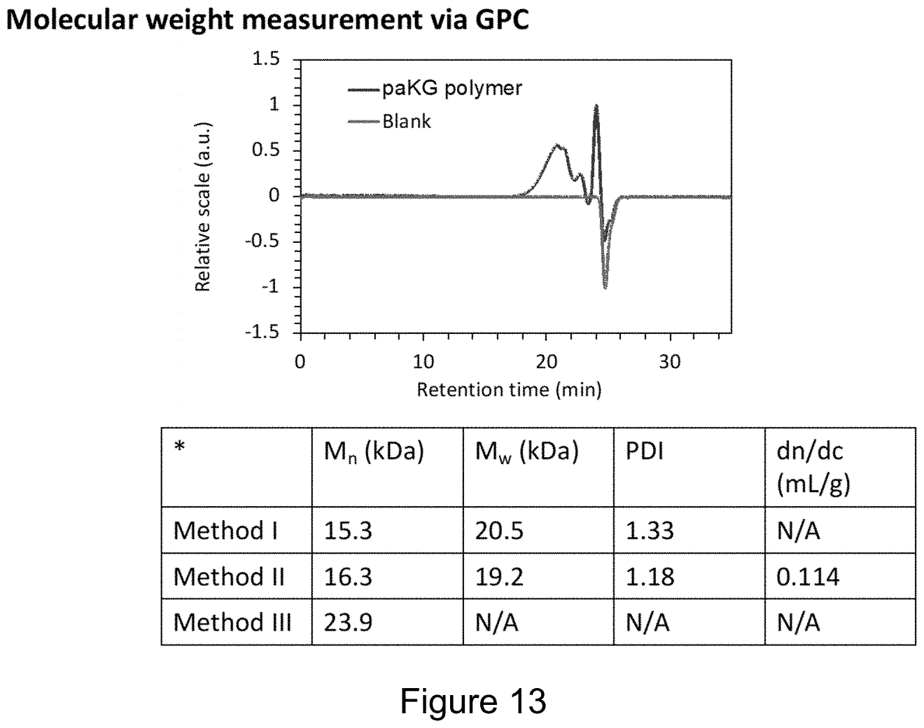

Steady state intracellular concentrations are needed to achieve the desired immunomodulation effect. Although, esterification of aKG molecules makes them cell permeable, aKG would need to be given in high and frequent dosages to achieve the desired effect in vivo. The high and frequent dosages are needed to overcome fast diffusion and elimination from the body caused by the low molecular weight. Therefore, polymers of aKG were generated, which can then degrade over a period of time to release aKG in a sustained manner after one-time application. Specifically, aKG was reacted with 1,10-decanediol to generate a polymer with a number-average molecular weight (Mₙ) of 15.3-23.9 kDa (FIG. 8A), which was determined using ¹H NMR spectroscopy and gel permeation chromatography (GPC) (FIG. 12 and FIG. 13). Polymers with octanediol and hexanediol were also generated, but led to low yields, and therefore 1,10-decanediol were used for further studies.

Next, in order to ensure that these polymers can deliver aKG intracellularly in phagocytes (e.g. DCs), these polymers were formulated into phagocytosable MPs using oil-in-water emulsions (DCs can phagocytose particles <15 μm (Champion & Mitragotri, Pharm. Res. 26(1):244-9, 2009)). The formation of particles was confirmed using scanning electron microscopy (SEM) and (FIG. 8B), the average size of these particles was determined to be 4 μm using dynamic light scattering (FIG. 8C), which matched SEM analyses. In order to test if these particles could release aKG in a sustained manner, release kinetics experiments in pH 7.4 (1× PBS, physiological pH) were performed. The amount of aKG released as a function of time was determined using high-performance liquid chromatography (HPLC), which demonstrates that the particles were able to release aKG in a sustained fashion for greater than 30 days. Cumulative release of aKG from PaKG MPs is shown in FIG. 8D.

### DCs are Capable of Phagocytosing PaKG MPs

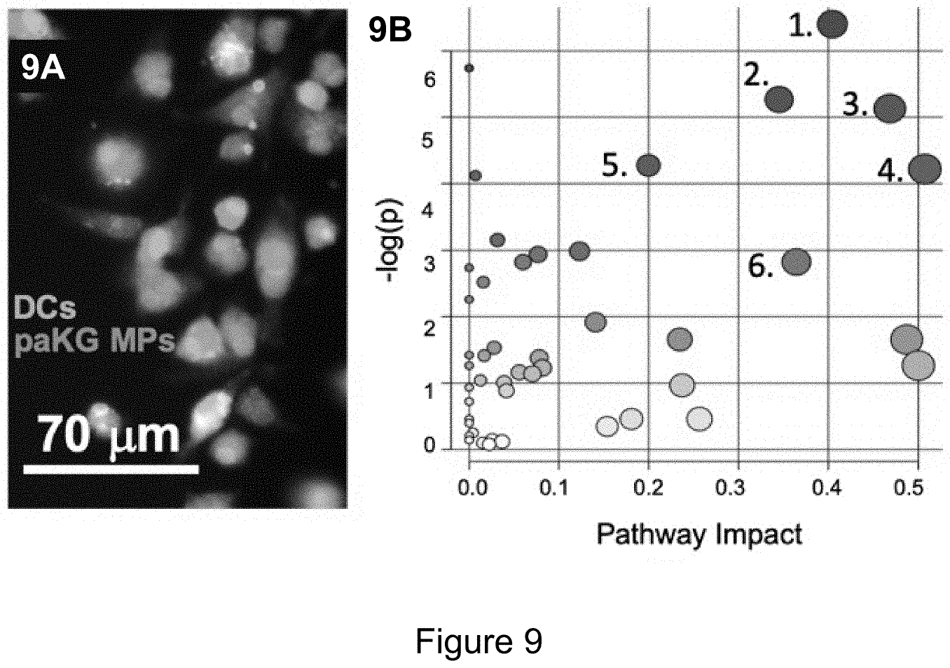

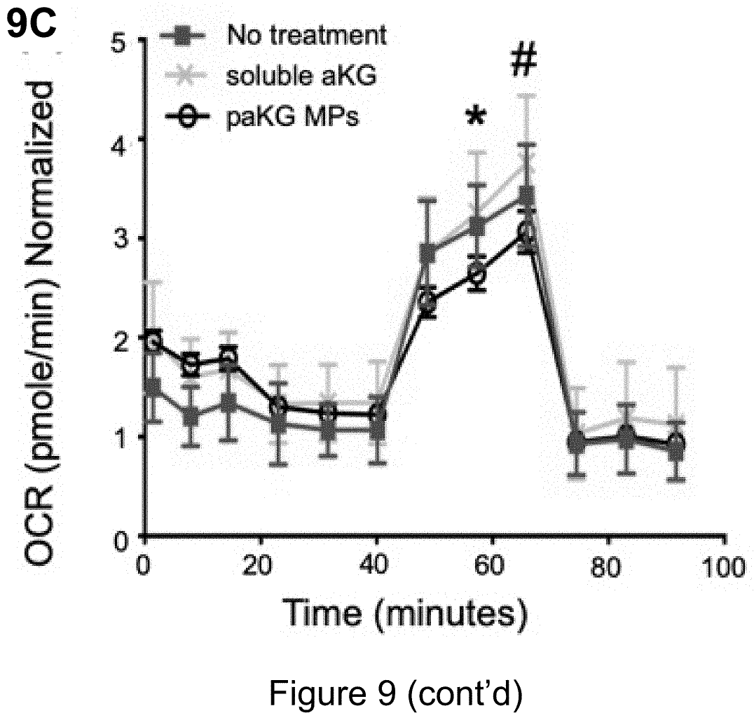

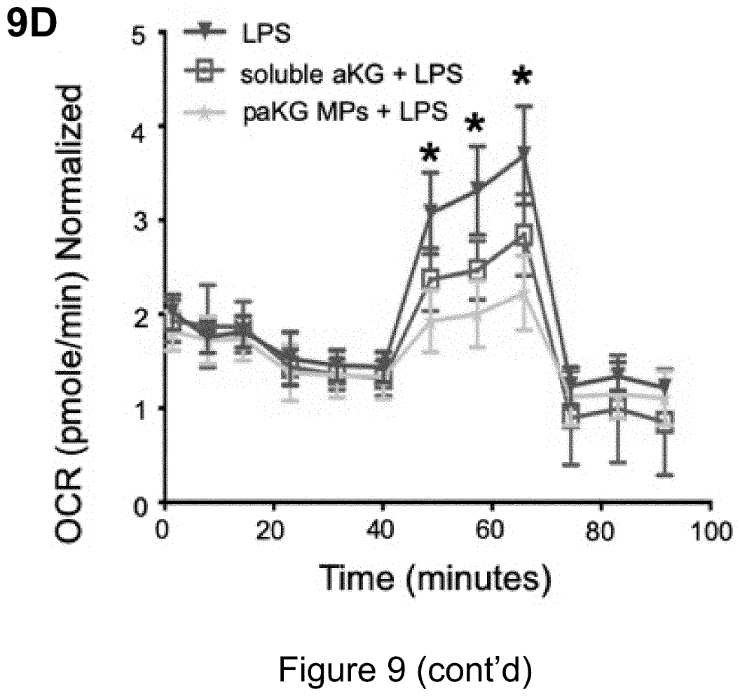

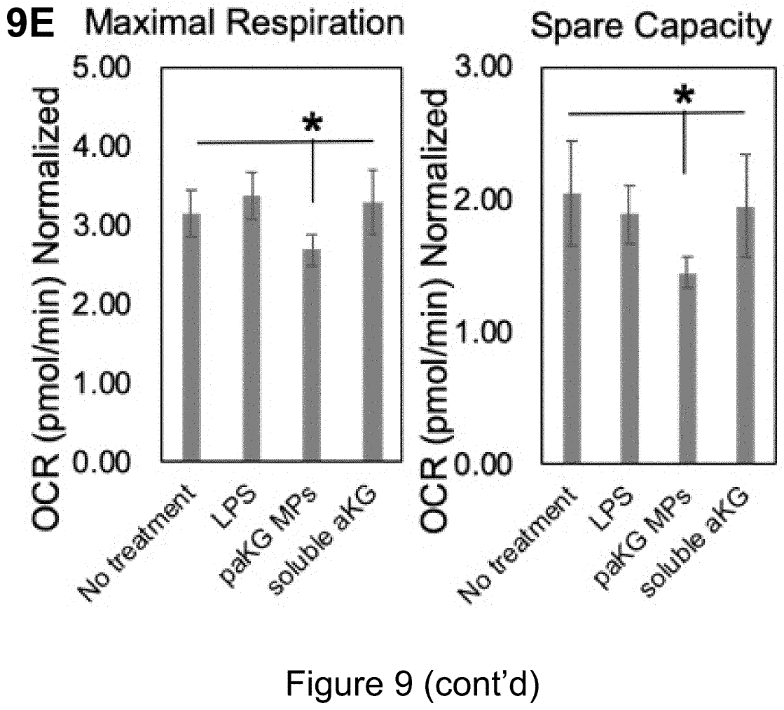

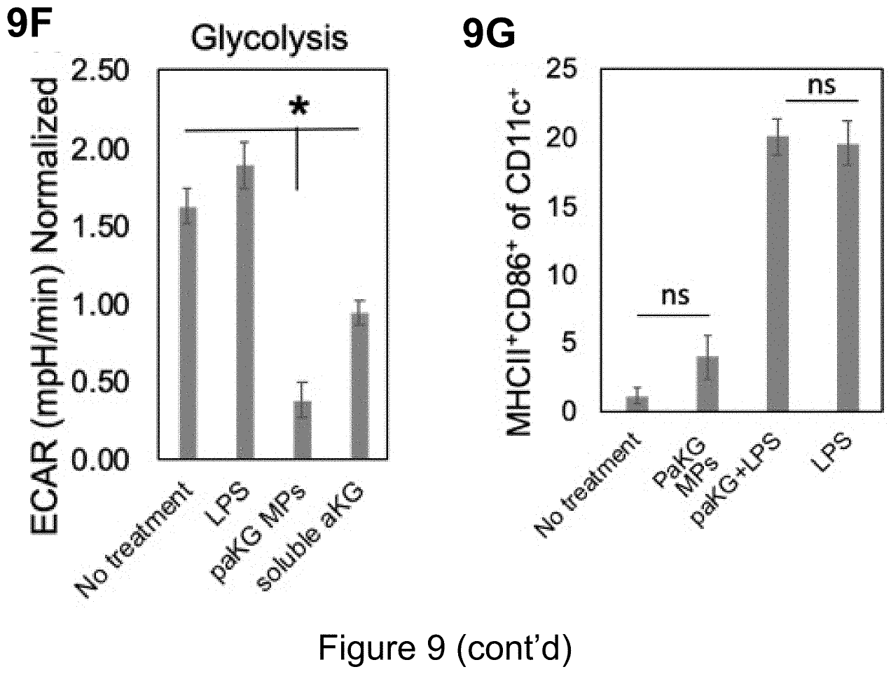

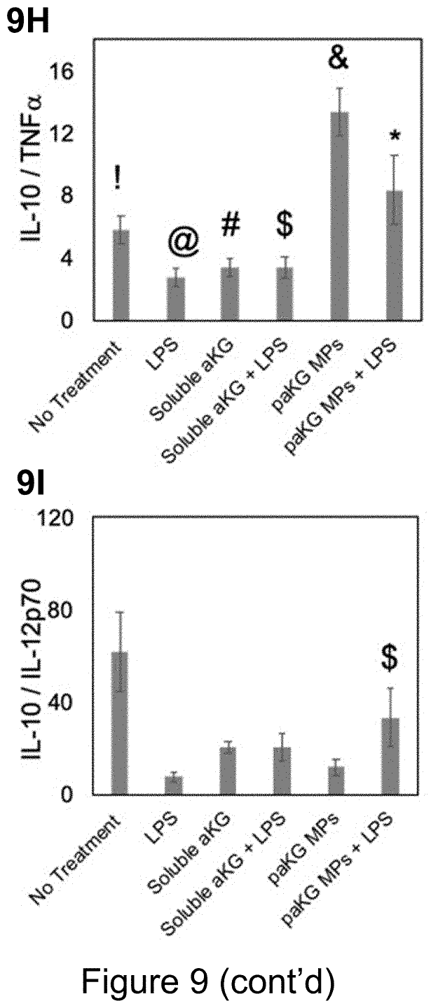

Although, DCs play an important role in modulating immune responses, the modulation in DC function after exposure to TCA metabolites is not well understood. In this study, the ability of PaKG MPs to deliver aKG was tested, potentially a key-immunosuppressive metabolite in TCA cycle, to DCs. In order for the intracellular sustained delivery of aKG to occur, DCs should be able to phagocytose the PaKG MPs. To determine if DCs are capable of phagocytosing PaKG MPs, bone marrow derived DCs were cultured for 60 minutes with Rhodamine 6G encapsulated PaKG MPs. As determined by immunofluorescence, it was observed that DCs were able to successfully phagocytose the PaKG MPs (FIG. 9A).

### PaKG MPs Modulate Intracellular Metabolite Profile in DCs

DC function can be modulated by changing the intracellular metabolite profile (Everts et al., Front. Immunol. 5:203, 2014). Since PaKG MPs release aKG in a sustained manner, the ability of these particles to modulate intracellular metabolite levels was measured using LC-MS (Shi et al., Anal. Chem. 91(21):13737-13745, 2019; Jasbi, et al., Proteome Res. 18(7):2791-2802, 2019; Parent et al., JAMA Surg. 151(7):e160853, 2016; Sood et al., Wound Repair Regen. 23(3):423-34, 2015; Carroll et al., Cancer Cell 27(2):271-85, 2015; Gu et al., Angew. Chemie Int. Ed. Engl. 55(50):15646-15650, 2016; Jasbi et al., Food Funct. 10(11):7343-7355, 2019). Modulation in intracellular metabolite levels in DCs was determined by culturing BMDCs with PaKG MPs. The intracellular metabolites were isolated, quantified using LC-MS/MS, normalized to protein amount, and the levels of these metabolites were compared to no treatment control.

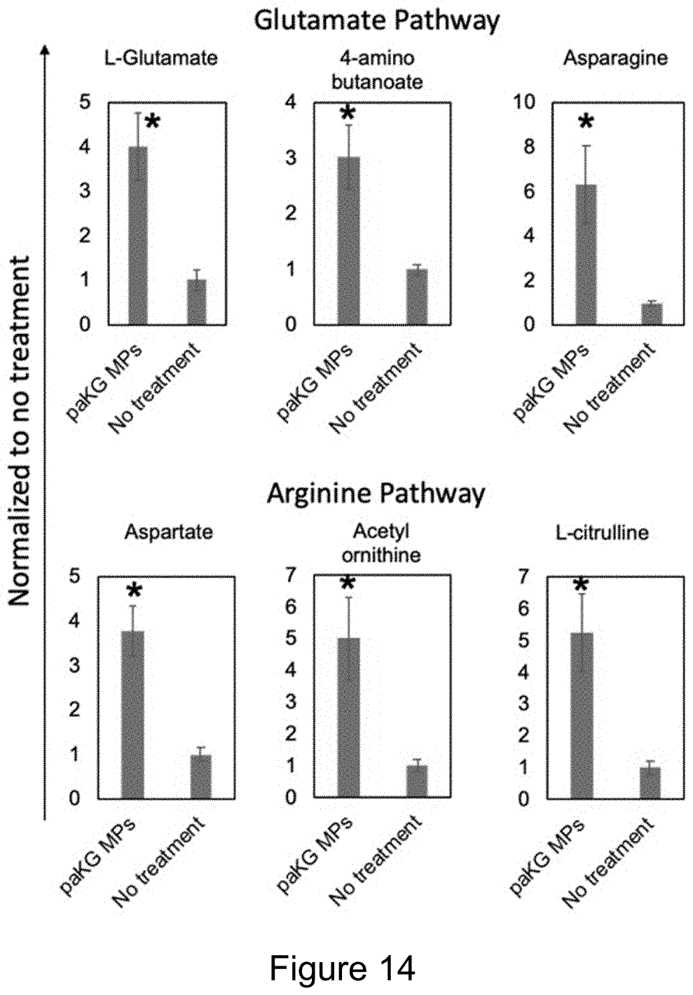

Enrichment analysis was conducted using the Kyoto Encyclopedia of Genes and Genomes (KEGG) database searches and metabolite intensities for different conditions was determined. The enrichment analysis of 299 reliably detected metabolites (using LC-MS) showed significant (p<0.05) perturbations or changes in the levels of glutamate, glycine/threonine/serine, alanine/aspartate/arginine and glutathione metabolisms, among others. Notably, large impact coefficient (>0.50) was observed in glutamate pathway suggesting the greatest impact of aKG delivery on the intracellular glutamate pathway metabolite levels (FIG. 9B). Specifically, L-glutamate, 4-aminobutanoate and asparagine in the glutamate pathway were 3- to 6-fold higher in DCs treated with PaKG MPs as compared to the no treatment control. Moreover, in the arginine pathway, aspartate, acetyl-ornithine and L-citrulline are upregulated in DCs treated with PaKG MPs approximately 4-fold as compared to the no treatment control (FIG. 14). Notably, these metabolites are involved in immune suppression via DCs (Bronte & Zanovello, Nat. Rev. Immunol. 5(8):641-54, 2005; Simioni et al., Int. J. Immunopathol. Pharmacol. 30(1):44-57, 2017; Rodriguez et al., Front. Immunol. 8:93, 2017).

Interestingly, although large impact coefficients were not observed in other metabolic pathways, certain metabolites involved in DC-mediated immunosuppression such as kynurenine (>6-fold increase), were significantly affected as compared to the no treatment. Moreover, the intracellular levels of aKG were not significantly different when compared to the no treatment control. Since glutamate and succinate were significantly upregulated, this suggests quick metabolization of aKG intracellularly. Overall, these data demonstrate that the PaKG MPs modulate intracellular metabolism of DCs

### PaKG MPs Reduce Glycolysis and Spare Capacity in DCs

Intracellular metabolite changes in DCs due to PaKG MPs suggest that the metabolic pathways in DCs might be modulated as well. Notably, upregulation of glycolysis and metabolic respiration are known to play an important role in DC activation (Kelly & O'Neill, Cell Res. 25(7):771-84, 2015). Therefore, in order to test if the PaKG MPs modulate glycolysis and metabolic respiration, Seahorse Assays were performed to determine the extracellular acidification rate (ECAR) and oxygen consumption rate (OCR). Lipopolysaccharide (LPS) was used as a positive control, and no treatment was used as a negative control. Additionally, extracellularly added soluble aKG to the cell culture was used as a control.

It was observed that the PaKG MPs have a lower trend of OCR as compared to the soluble aKG and no treatment groups. Importantly, in the presence of LPS (mimicking inflammation e.g. in wound healing), PaKG substantially decreased the OCR in DCs, suggesting substantial decrease in the metabolic respiration (FIG. 9C and FIG. 9D). In order to further analyze this data, basal respiration without glucose, maximal respiration in the presence of glucose, and spare capacity (determines the ability of the cell to respond to an energetic demand) was determined. It was observed that the DCs treated with PaKG MPs had higher basal respiration, lower maximal respiration and lower spare capacity than the no treatment control (FIG. 9E).

In addition, glycolysis was significantly decreased in DCs cultured with PaKG MPs as compared to LPS (positive control) and no treatment (negative control). Interestingly, soluble aKG decreased glycolysis in DCs compared to the no treatment control; however, soluble aKG had higher glycolysis levels in DCs as compared to PaKG MPs (FIG. 9F). Taken together, these data demonstrate that the PaKG MPs reduce the metabolic activity of DCs even in the presence of LPS. Thus, these data suggest that PaKG MPs might be beneficial in preventing uncontrolled inflammation due to reduced DC metabolic activity.

### PaKG MPs Do Not Activate DCs In Vitro

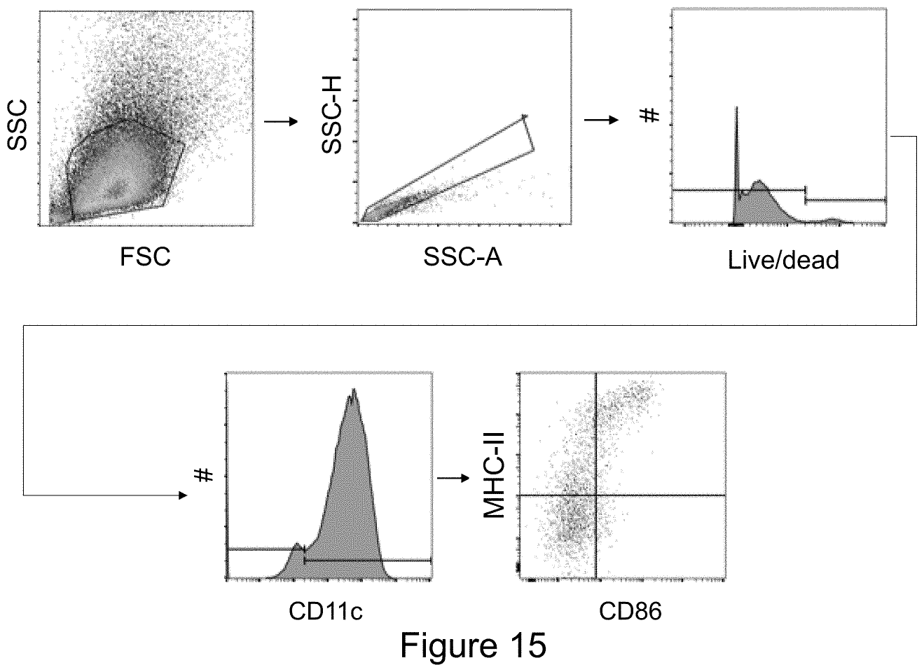

The metabolism of DCs can play an important role in its function including changes in MHC-II, CD86 expression, cytokine production and adaptive immune responses (O'Neill & Pearce, J. Exp. Med. 213(1):15-23, 2016; Everts et al., Front. Immunol. 5:203, 2014; Kelly & O'Neill, Cell Res. 25(7):771-84, 2015). In order to test if PaKG MPs modulated DC metabolism, can modify its function, MHC-II, CD86, IL-10, IL-12p70 and TNF-alpha expression was determined using flow cytometry (FIG. 15).

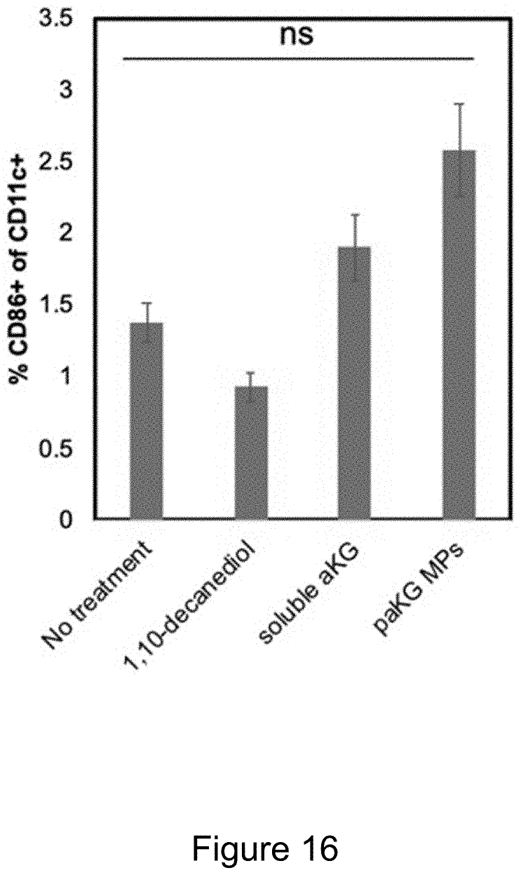

It was observed that the PaKG MPs do not activate DCs as compared to untreated immature DCs, since frequency of MHC-II⁺CD86⁺ was not significantly different from each other. On the other hand, LPS (positive control), significantly upregulated the frequency of MHC-II⁺CD86⁺ in DCs (FIG. 9G). Additionally, the controls of 1,10-decanediol, and soluble aKG, added at the equivalent amount as the PaKG MPs did not significantly change the CD86 expression in CD11c+ cells as compared to the no treatment control and the PaKG MP condition (FIG. 16). Moreover, characterization of anti-inflammatory IL-10, pro-inflammatory TNF-alpha and pro-inflammatory IL-12p70 expression demonstrated that PaKG MPs with or without LPS were able to significantly upregulate IL-10/TNF-alpha ratio as compared to all other conditions (FIG. 9H). Moreover, PaKG MPs in the presence of LPS, significantly increased the IL-10/IL-12p70 ratio as compared to PaKG MPs alone and LPS alone conditions, and this increase was not significantly different than the no treatment control (FIG. 9I). These data suggest that DCs have an immunosuppressive phenotype in the presence of PaKG MPs, which combined with the surface expression of MHC-II and CD86 may modulate adaptive immune responses.

### PaKG MPs Decreases Pro-Inflammatory T Cell Responses in Allogeneic Mixed Lymphocyte Reaction

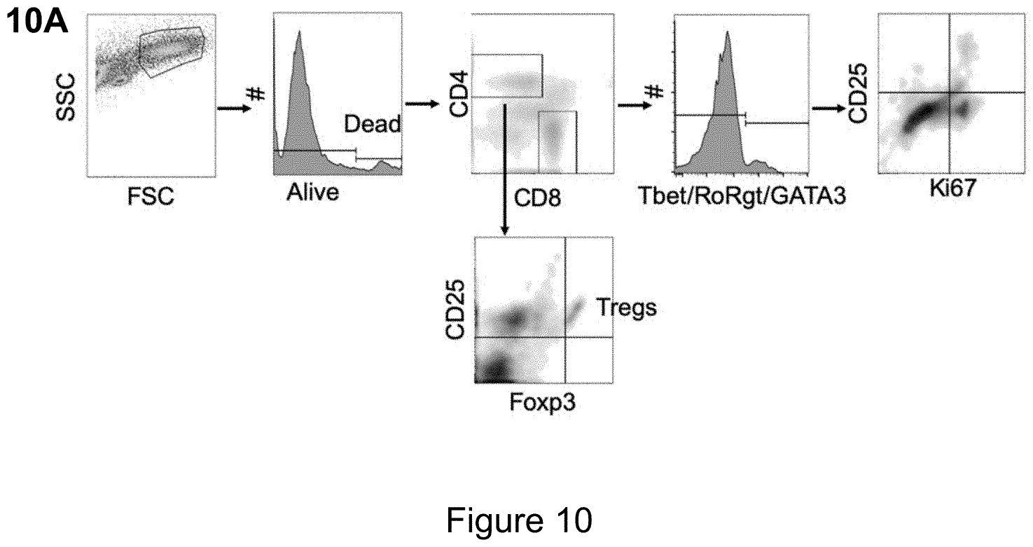

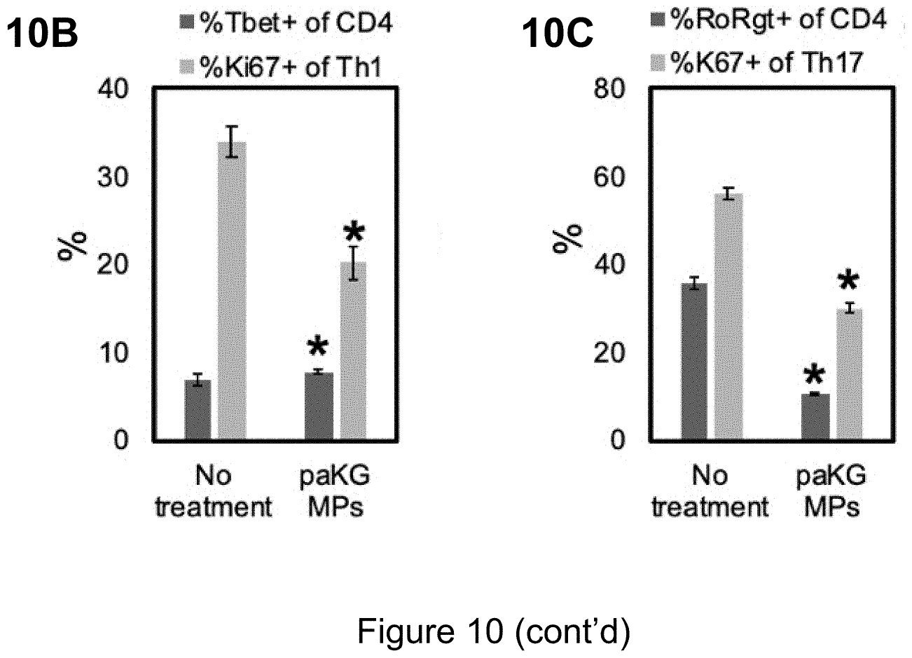

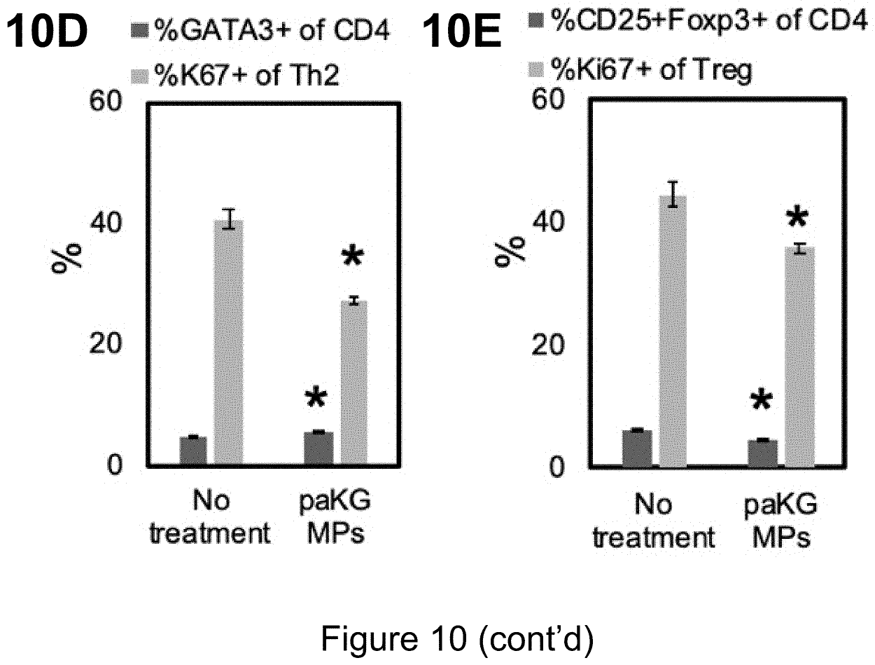

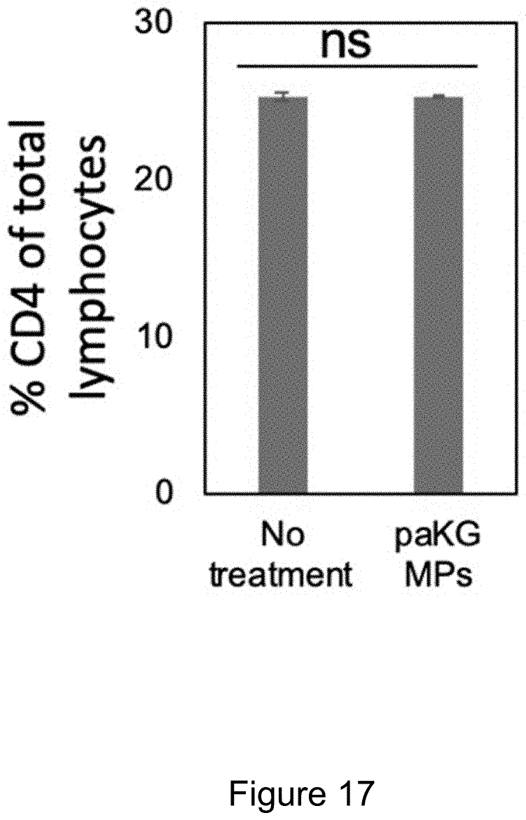

DCs are effective modulators of T cell responses, which are essential for generating adaptive immune responses. PaKG MPs modulate DC phenotype and cytokine production, which might also modulate T cell responses. Therefore, a mixed lymphocyte reaction (MLR) with BALB/c mice-derived CD3⁺ T-cells and C57BL/6j mice-derived DCs were cultured with the PaKG MPs. Co-culture of untreated DCs and T cells was used as control. These cells were cultured for 48-72 hours and the frequency of T helper type 1 (Th1−CD4⁺Tbet⁺), Th2 (CD4⁺GATA3⁺), Tc1 (CD8⁺Tbet⁺) and Treg (CD4⁺CD25⁺Foxp3⁺), along with proliferation (Ki67⁺) and activation (CD25⁺) in these cells (FIG. 10A) was determined. The DCs that were treated with PaKG MPs down-regulated Th1, Th2, Th17 and Treg population proliferation in the MLR (FIGS. 10B-10E). Notably, the decrease in the proliferation of Th1 and Th17 population was substantially higher as compared to Treg population decrease, suggesting preferential decrease in pro-inflammatory cell type. Additionally, it was observed that the PaKG MPs did not modulate the frequency of CD4⁺ T cell percentage in the MLR (FIG. 17) as compared to the no treatment control. These data strongly suggest that PaKG MPs by themselves may prevent CD4⁺ pro-inflammatory immune activation.

### PaKG MPs Application on Cutaneous Wounds Lead to Faster Wound Closure

Immunosuppression has been shown to accelerate wound healing (Bootun, Int. Wound J. 10(1):98-104, 2013). Since, PaKG MPs induce an immunosuppressive response, the ability of these particles to induce accelerated wound healing was tested in a mouse model of cutaneous wound.

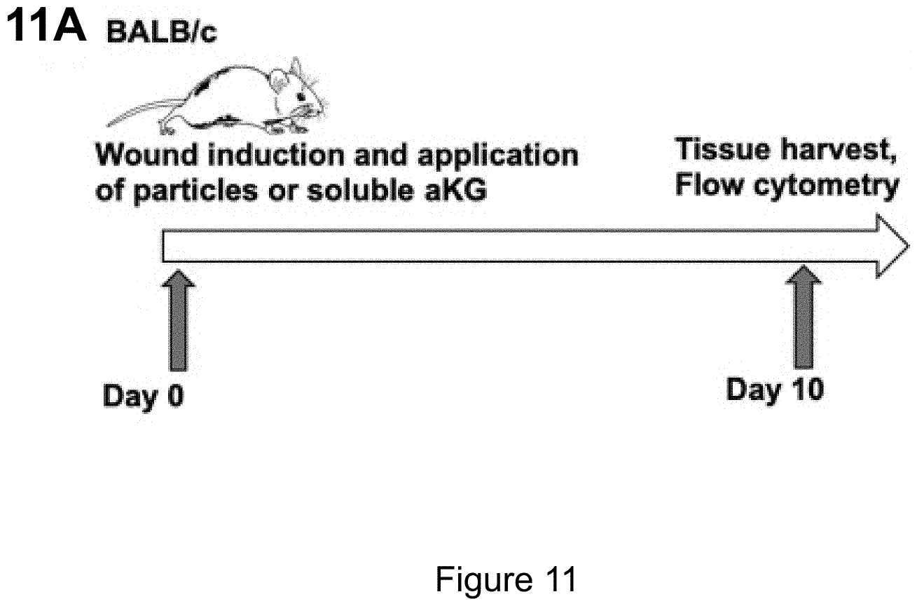

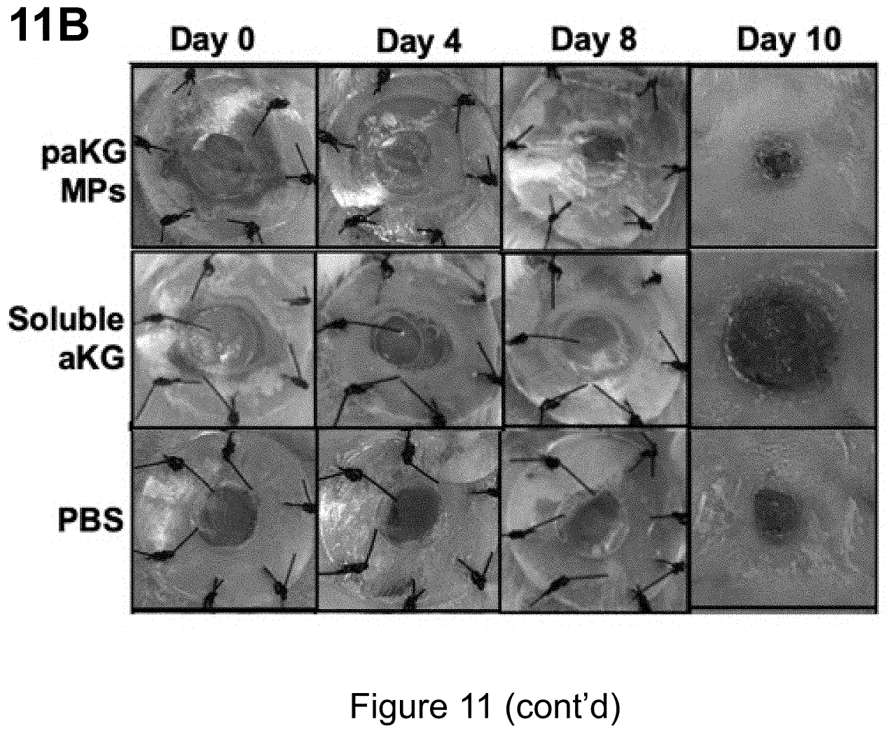

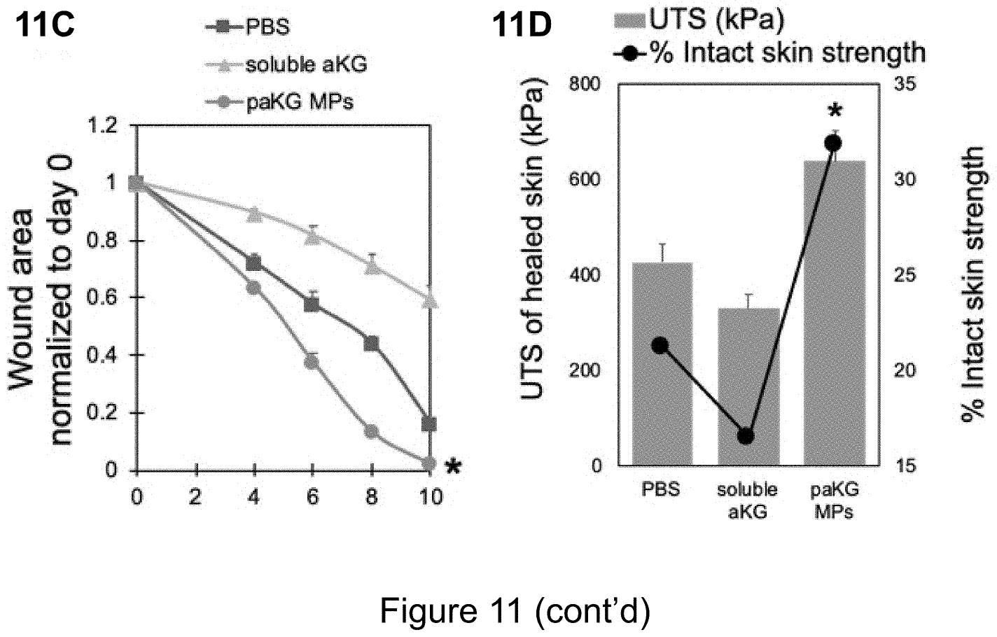

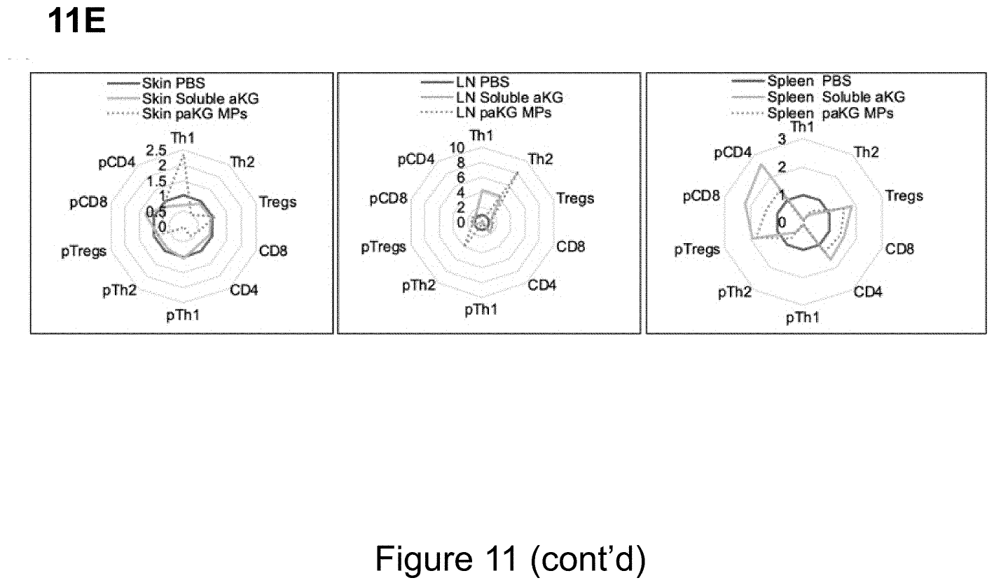

Cutaneous wounds with splints were created in immunocompetent BALB/c mice (FIG. 11A) and PaKG MPs or saline or soluble aKG were applied on top of the wound on day 0, in addition to a Tegaderm dressing. Wound closure was observed for 10 days by taking photographs and determining wound area measurements with Image J software (FIG. 11B and FIG. 11C). As determined by the photographic representations, the PaKG MP group fully healed while the control wounds were not fully healed. On day 10, mice were sacrificed and draining inguinal lymph nodes, spleen and wounded skin were isolated. Ultimate tensile strength (UTS) studies were performed on the skin to determine the strength of the healed skin. The UTS was significantly higher in the PaKG MP group as compared to the saline control (FIG. 11D). Importantly, it was observed that the wounds closed at day 10 in PaKG MP group, which was significantly faster as compared to soluble aKG and saline.

### PaKG MPs Lead to Accelerated Wound Healing by Upregulating Th2 Population in the Draining Lymph Nodes

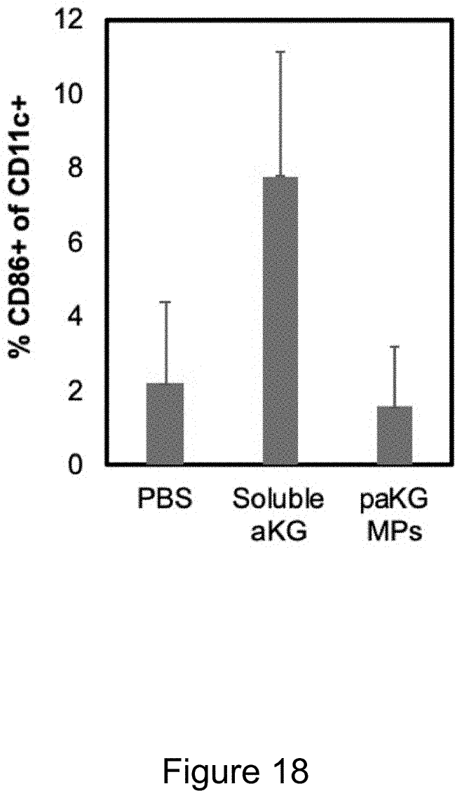

Phagocytic cells and Th2 cells cross-talk to generate wound healing responses. Therefore, on day 10, the frequency of T cells in the draining lymph nodes was analyzed. Interestingly, the Th2 and proliferating Th2 population was significantly upregulated in PaKG MPs, as compared to PBS and soluble aKG in the draining lymph nodes (FIG. 11E). Moreover, it was observed that the PaKG MPs induced higher Th1 population in the skin. However, the proliferating Th1 population was significantly lower than PBS and soluble aKG (FIG. 11E). PaKG MPs also demonstrated lower trends of DC activation (% CD86⁺ of CD11c⁺) cells in the skin post healing (FIG. 18), as compared to the controls. Lastly, the PaKG MPs had significantly lowered levels of proliferating T cells in the spleen as compared to soluble aKG, which suggests lowered systemic responses as compared to soluble aKG delivery (FIG. 11E). Taken together these data suggest that the PaKG MPs potentially accelerated wound healing by locally upregulating proliferating Th2 cell populations, while reducing systemic immune modulation.

### Metabolite-Based Polymers Provide a New Technique to Deliver Metabolites Intracellularly and Locally to Modulate the Metabolism of Immune Cells

The data presented herein demonstrates for the first time. that one-time application of aKG metabolite-based polymeric microparticles can lead to modulation of the immune system. Moreover, this study also demonstrates that aKG is an immunosuppressive metabolite that can control the function of innate and adaptive immune responses in vitro and in vivo.

Metabolic reprogramming can orchestrate immune cell polarization and contribute to functional plasticity (O'Neill & Pearce, J. Exp. Med. 213(1):15-23, 2016; Van den Bossche et al., Cell Rep. 17(3):684-696, 2016; Pearce & Everts, Nat. Rev. Immunol. 15(1):18-29, 2015). Notably, it has been demonstrated that immune cells can generate an anti-inflammatory response when cell permeable aKG is delivered to macrophages (Liu et al., Nat. Immunol. 18(9):985-994, 2017). However, delivery of these molecules in vivo can be challenging due to the quick diffusion (within seconds) of small molecules in vivo (Sun et al., ACS Nano, 10(7):6769-81, 2016). Therefore, in this work, a polymer made of aKG metabolite was generated that could release aKG in a sustained manner. This is the first evidence of metabolite-based polymers that can be used to deliver metabolites in a sustained fashion.

Metabolism modulating enzyme inhibitor drugs can also be used to alter the function of macrophages and DCs, and thus modulate disease outcomes. For example, global immune suppression can be achieved using glycolytic inhibitors 2-deoxyglucose (Abboud et al., Front. Immunol. 9:1973, 2018). However, these inhibitors can also lead to prevention of proliferation and migration of endothelial cells and hence may not be suitable for applications such as wound healing (Falkenberg et al., Nat. Metab. 1(10):937-946 2019). Therefore, development of a strategy that does not modulate the glycolytic enzymes but still leads to local suppression of immune cells can be beneficial for wound healing.

Targeting phagocytic cells (e.g. macrophages, DCs) in a tissue, where there is a diverse type of cell population is challenging. Therefore, microparticles made of the aKG polymer that can be picked or phagocytosed by immune cells, were utilized in this study. Interestingly, these PaKG MPs also provide an added advantage of encapsulating and delivering drugs intracellularly in phagocytic cells, which was demonstrated by delivering rhodamine as a representative fluorescent drug molecule dye. This strategy can further be utilized to deliver proteins, peptides or other drugs that can then modulate the function of these phagocytic cells.

In a cutaneous wound, distress to the upper layer of the skin, the epidermis, can trigger a series of cellular and humoral immune responses that involve the recruitment of immune cells to the wound bed. Specifically, innate immune cells such as DCs, which are professional APCs, prevent pathogens from entering the wound bed (Keyes et al., Cell 167(5):1323-1338, 2016). Importantly, DCs form a bridge between the innate and adaptive immune system and modulate the function of lymphocytes such as T cells (Acharya et al., Adv. Funct. Mater. 27(5): 1604366, 2017; Acharya et al., Biomaterials 30(25):4168-77, 2009). T cells play a crucial role in defending against pathogenic invaders and are involved in both the inflammatory and remodeling phase in wound healing (Havran & Jameson, J. Immunol. 184(10):5423-8, 2010). Following the inflammatory phase, the wound bed undergoes a phase of remodeling and repair. Interestingly, although growth factors act during the remodeling and repair phase to improve wound healing kinetics, these do not directly modulate the inflammatory or anti-inflammatory phase of the wound healing process, and therefore, are known to be sub-optimal (Olekson et al., Wound Repair Regen. 23(5):711-23, 2015; Yeboah et al., Adv. Wound Care 1; 6(1):10-22, 2017; Panoskaltsis-Mortari et al., Am J Physiol Lung Cell Mol Physiol 278(5):L988-99, 2000). The data presented herein demonstrate that PaKG MPs might assist the wound closure by accelerating the anti-inflammatory response by releasing aKG intracellularly or extracellularly in a sustained manner. In contrast, the cutaneous wounds that were exposed to soluble aKG received aKG in a bolus manner, leading to immediate immune suppression and, thereby, surpassing the inflammatory phase of wound healing. Hence, soluble aKG might prohibit professional APCs from eliminating pathogens in the wound bed, while the sustained release of aKG from the PaKG MP still allowed for the inflammatory phase to occur but potentially accelerated the remodeling phase.

The cross-talk between phagocytic cells and T cells provide signals toward healing responses in vivo (Keyes et al., Cell 167(5):1323-1338, 2016; Havran & Jameson, J. Immunol. 184(10):5423-8, 2010; Sadtler et al., Science 352(6283):366-70, 2016; Swift et al., J. Invest. Dermatol. 117(5):1027-35, 2001). Interestingly, Th2 cells, by secreting IL-4, may induce faster wound healing responses (Sadtler et al., Science 352(6283):366-70, 2016). In these studies, although PaKG MPs did not upregulate Th2 frequency in vitro in the allogenic mixed lymphocyte reactions, the Th2 frequency in the draining lymph nodes was higher in the PaKG MPs group as compared to the controls at the wound closure time point. Importantly, the CD4⁺ and CD8⁺ T cell proliferation was lower in PaKG MPs as compared to soluble aKG, while Treg proliferation was similar in these two conditions. Interestingly, the T cell population was less affected in the spleen in PaKG MPs condition as compared to the soluble aKG, which suggests that at the wound closure stage the systemic population is not affected by the PaKG MPs. Overall, these data suggest that the PaKG MPs might be inducing accelerated wound closure via upregulating Th2 frequency locally, while upregulating or maintaining Treg population systemically.

In summary, metabolite-based polymers provide a new technique to deliver metabolites intracellularly and locally to modulate the metabolism of immune cells. In this study, PaKG MPs were generated that could not only modulate DC function but also modulate T cell functions in vitro as well as in vivo. Importantly, PaKG MPs were able to provide accelerated wound closure responses in vivo by generating an immunosuppressive microenvironment

### Polymer Synthesis and Characterization

Ketoglutaric acid and 1,10-decanediol were mixed at equimolar ratio in a round-bottom flask. This mixture was stirred at 130° C. for 48 hours under nitrogen. The polymer thus generated was precipitated in methanol solution, and the unreacted monomers were removed by multiple (3×) precipitation steps. Residual methanol was then evaporated off using a rotary evaporator, and the polymers were then dried under vacuum at room temperature for 48 hours.

¹H-NMR spectroscopy was performed on a Varian 500 MHz spectrometer using deuterated chloroform (CDCl₃), with a concentration of 5 mg/ml. All ¹H NMR experiments are reported in δ or parts per million (ppm) unit and were measured relative to the chloroform H-signal (7.26 ppm) in CDCl₃, unless stated differently. The following abbreviations were used to indicate multiplicities: s (singlet), d (doublet), t (triplet), q (quartet), m (multiplet), br (broad), and tt (triplet of triplets). Coupling constants are expressed in hertz (Hz). ¹H NMR (500 MHz, Chloroform-d) δ4.21 (t, 82H), 4.03 (t, 85H), 3.11 (t, 88H), 2.94 (t, 1H), 2.62 (t, 88H), 1.68 (tt, 91H), 1.57 (tt, 91H), 1.25 (br, 571H).

The molecular weight of the polymer was determined using GPC and ¹H NMR spectroscopy. GPC was performed using a Waters Alliance e2695 HPLC system interfaced to a light scattering detector (miniDAWN TREOS) and an Optilab T-rEX differential refractive index (dRI) detector controlled using Astra v6.1 software. The mobile phase was tetrahydrofuran (THF) Optima (inhibitor-free) at a flow rate of 1.0 mL/min and molecular weights were determined either via a calibration curve prepared using Agilent low dispersity polystyrene standards of 500, 200, 100, 30, 10, and 5 kDa or by determining the refractive index increment using the RI detector and using the light scattering detector response to determine an absolute weight-average molecular weight (M_{w}). The PaKG samples were dissolved in THF at ˜1.0 mg/mL and passed through 0.22 μm filters before injection to the GPC system. For molecular weight determination by ¹H NMR spectroscopy, the integration of the peaks attributed to the end group protons were compared to the integrations of the peaks from the main-chain backbone protons to determine the number-average molecular weight (Mₙ).

### Microparticle Synthesis and Characterization

PaKG polymers were utilized to generate microparticles using a water-oil-water emulsion method. In order to generate microparticles, first, 50 mg of the polymers were dissolved in 1 mL of dichloromethane (DCM). This solution was then added to 10 mL of 2% polyvinyl alcohol (PVA) solution in deionized water (DIH₂O) and homogenized at 30,000 rpm using a handheld homogenizer for 2 minutes. This emulsion was then added to 50 mL of 1% (vol/vol) PVA solution and stirred at 400 rpm for 3 hours to remove DCM. The particles thus formed were then washed 3 times by centrifuging at 2000 × g for 5 minutes, removing the supernatant and resuspending in DIH2O. These particles were then freeze dried and used for next experiments.

The particles were imaged using a scanning electron microscope (SEM) XL30 Environmental FEG-FEI. Moreover, the size of the particles was quantified using dynamic light scattering.

Release kinetics of the metabolites were determined by incubating 1 mg of the microparticles in 1 mL of phosphate buffered saline (PBS) and placed on a rotator at 37° C. Next, triplicates of each release sample were centrifuged at 2000 × g for 5 minutes. After centrifugation, 800 μL of the supernatant was removed and placed into a separate 1.5 mL tube and then replaced by 800 μL of new buffer.

The amount of metabolite released was then determined by developing a new method in a high-performance liquid chromatography (HPLC). Specifically, the mobile phase of 0.02 M H₂SO₄ in water was used. A 50 μL of injection volume was utilized in a Hi-Plex H, 7.7×300 mm, 8 μm column. A flow rate of 1.2 mL/min was utilized and the absorbance was determined using a UV detector at 210 nm. The area under the curve was determined in order to determine the concentration using ChemStation analysis software.

### Dendritic Cell Isolation and Culture

Immature bone marrow-derived DCs were generated from 6-8 week-old female C57BL/6j mice using a modified 10-day protocol (Acharya et al., Adv. Funct. Mater. 27(5): 1604366, 2017; Acharya et al., Biomaterials 29(36):4736-50, 2008; Acharya et al., Acta Biomater. 7(1):180-92, 2011). Briefly, femur and tibia from mice were isolated and kept in wash media composed of DMEM/F-12 (1:1) with L-glutamine, 10% fetal bovine and 1% penicillin-streptomycin. The ends of the bones were cut, and bone marrow was flushed out with 10 mL wash media and mixed to make a homogeneous suspension. Red blood cells were lysed by incubating and resuspending the pellet in 3 mL 1× red blood cell (RBC) lysis buffer for 3 minutes at 4° C. The cell suspension was then washed twice with wash media and re-suspended in DMEM/F-12 with L-glutamine, 10% fetal bovine serum, 1% sodium pyruvate, 1% non-essential amino acids (VWR, Radnor, Pa.), 1% penicillin-streptomycin and 20 ng/ml GM-CSF (DC media). All % values shown here are vol/vol. This cell suspension was then seeded in a tissue culture-treated T-75 flask (day 0). After 48 hours (day 2), floating cells were collected, centrifuged, re-suspended in fresh media and seeded on low attachment plates for 6 additional days. Half of the media was changed every alternate day. At the end of 6 days (day 8), cells were lifted from the low attachment wells by gentle pipetting, re-suspended and seeded on tissue culture-treated polystyrene plates for 2 more days before treating them. On day 10, cells were treated with either 0.1 μg/ml of PaKG MPs or 0.1 μg/mL of soluble aKG and LPS conditions were administered 1 μg/mL of LPS. Purity, yield, and immaturity of DCs (CD11c, MHC-II and CD86) were verified via immunofluorescence staining and flow cytometry. Dendritic cells were isolated from at least 3 separate mice for each experiment.

### Mixed Lymphocyte Reactions

Spleens were isolated from 6-8-week-old BALB/c mice. Single cell suspensions were prepared by mincing the spleen through a 70 μm pore sized cell strainer. The effluent was centrifuged for 5 minutes at 300 × g. The cells were then resuspended in 3 mL 1× RBC lysis buffer for 3 minutes at 4° C. Next the cells were spun down at 300 × g for 5 minutes and the pellet was re-suspended in 4 μL of buffer (0.5% BSA and 2 mM EDTA in PBS) per million cells. Negative selection of CD3⁺ T-cells was performed according to manufacturer's recommendation. A biotin-labeled antibody cocktail (CD8a (Ly-2) (rat IgG2a), CD11b (Mac-1) (rat IgG2b), CD45R (B220) (rat IgG2a), DX5 (rat IgM) and Ter-119 (rat IgG2b)) was added (10 μL per 10 million cells) and incubated for 15 minutes at 4° C. Buffer (30 μL) and anti-biotin microbeads (10 μL) were added to the mixture per 10 million cells. After 15 minutes incubation at 4° C., cells were centrifuged at 300 × g for 5 minutes and re-suspended in 500 μL of buffer per 100 million cells. A magnetic column was then utilized to collect CD3⁺ T-cells. The CD3⁺ T-cells were centrifuged at 300 × g for 5 minutes and used in mixed lymphocyte reaction. Bone marrow derived DCs were isolated from C57BL/6j mice and were treated prior to the addition of T cells. Mixed lymphocyte reaction was performed at DC:T cell ratio of 1:5 for 48-72 hours.

### LC-MS Metabolomics Studies

Bone marrow derived DCs from C57BL/6j were cultured in 6 well plates at 1 million cells per well. PaKG MPs were added at 50 μg/well and no treatment was utilized as a control. After 24 hours of culture, the supernatant was removed, and the cells were gently rinsed with 2 mL of 37° C. PBS. Next, immediately, 1 mL of 80:20 methanol:H₂O (−80° C.) into the plates, and the plates were then placed on dry ice to quench metabolism and perform extraction. After 30 minutes of incubation on dry ice the cells were scraped using a cell scraper and transferred into centrifuge tubes. The tubes were then spun at 16,000 rpm for 5 minutes at 4° C. The soluble extract was removed into a vial and completely dried. The pellets were utilized to measure the total protein using Nanodrop 2000.

The LC-MS/MS method was performed according to previous protocols (Shi et al., Anal. Chem. 91(21):13737-13745, 2019; Jasbi, et al., Proteome Res. 18(7):2791-2802, 2019; Parent et al., JAMA Surg. 151(7):e160853, 2016; Sood et al., Wound Repair Regen. 23(3):423-34, 2015; Gu et al., Angew. Chemie Int. Ed. Engl. 55(50):15646-15650, 2016; Jasbi et al., Food Funct. 10(11):7343-7355, 2019). Briefly, LC-MS/MS were performed using Agilent 1290 UPLC-6490 QQQ-MS system. A total of 10 μL of the processed samples were injected twice, for analysis using negative ionization mode and a total of 4 μL of the processes sample for analysis using positive ionization mode. Both chromatographic separations were performed in hydrophilic interaction chromatography (HILIC) mode on a Waters XBridge BEH Amide column (150×2.1 mm, 2.5 μm particle size). The flow rate utilized in these studies was 0.3 mL/min, auto-sampler temperature was kept at 4° C., and the column compartment was set at 40° C. The mobile phase was composed of Solvents A—10 mM ammonium acetate, 10 mM ammonium hydroxide in 95% H₂O/5% ACN and Solvent B—10 mM ammonium acetate, 10 mM ammonium hydroxide in 95% acetonitrile (ACN)/5% H₂O. After the initial 1 minute isocratic elution of 90% B, the percentage of Solvent B decreased to 40% at t=11 minutes. The composition of Solvent B maintained at 40% for 4 minutes (t=15 minutes), and then the percentage of B gradually went back to 90% to prepare for the next injection.

The mass spectrometer is equipped with an electrospray ionization (ESI) source. Targeted data acquisition was performed in multiple-reaction-monitoring (MRM) mode. A total of ˜320 MRM transitions in negative and positive modes were observed. The whole LC-MS system was controlled by Agilent Masshunter Workstation software. The extracted MRM peaks were integrated using Agilent MassHunter Quantitative Data Analysis.

### Seahorse Assay

Glycolysis and oxidative phosphorylation were measured with the Seahorse Extracellular Flux XF-96) analyzer as previously described (Curtis et al., 29(1):141-155, Cell Metab. 2019). Briefly, cells were seeded in Seahorse XF-96 plates at a density of 50,000 cells per well and cultured for 24 hours in the presence of 10 μg/well of PaKG MPs or equivalent amount of soluble aKG, in the presence of absence of 1 μg/mL of LPS. After 24 hours, cells were changed to unbuffered DMEM in the absence of glucose. Sequential injections were performed with D-glucose (10 mM), oligomycin (1 mM), and 2-deoxyglucose (100 mmol/L). The extracellular acidification rates (ECAR) after the injection of D-glucose was a measure of glycolysis, and the ECAR after the injection of oligomycin represented maximal glycolytic capacity. Non-glycolytic activity was quantified by the measure of ECAR after the injection of 2-deoxyglucose. Samples were analyzed with 10 technical replicates.

For oxidative phosphorylation, 24 hours after cell seeding, media was changed to unbuffered DMEM containing 2 mM glutamine, 1 mM pyruvate, and 10 mM glucose. Sequential injections were performed with oligomycin (2 mM), 7 Carbonyl cyanide-4 (trifluoromethoxy) phenylhydrazone (FCCP) (1 mM), and antimycin/rotenone (1 mM) to modify mitochondrial membrane potential. The oxygen consumption rate (OCR) after the injection of oligomycin was a measure of ATP-linked respiration and the OCR after the injection of FCCP represented maximal respiratory capacity. Basal respiration was quantified by the measure of OCR prior to the injection of oligomycin. Samples were analyzed with 10 technical replicates.

### Flow Cytometry

All the antibodies were purchased and used as is. Flow staining buffers were prepared by generating 0.1% bovine serum albumin, 2 mM Na₂EDTA and 0.01% NaN₃. Live/dead staining was performed using fixable dye eF780. Flow cytometry was performed by following manufacturer's recommendations using Attune NXT Flow cytometer. The antibodies used in these studies are shown in Table 1.

TABLE 1
Antibodies

| | Target | Fluorophore | Company | Catalog # | Clone |
|---|---|---|---|---|---|
| 1 | CD4 | BB700 | BD Biosciences | 566407 | RM4-5 |
| 2 | CD8 | APC-R700 | BD Biosciences | 564983 | 53-6.7 |
| 3 | CD25 | PECy7 | BD Biosciences | 552880 | PC61 |
| 4 | CD11c | PE | BioLegend | 117308 | N418 |
| 5 | CD86 | SB600 | ThermoFisher | 63-0862-82 | GL1 |
| | | | Scientific | | |
| 6 | MHC | APC | BioLegend | 107614 | M5/114.15.2 |
| 7 | Tbet | BV785 | BioLegend | 644835 | 4B10 |
| 8 | FoxP3 | eF450 | Invitrogen | 48-5773-82 | FJK-16s |
| 9 | RORγT | BV650 | BD Biosciences | 564722 | Q31-378 |
| 10 | Ki67 | FITC | Invitrogen | 11-5698-82 | SolA15 |
| 11 | GATA3 | BV711 | BD Biosciences | 565449 | L50-823 |
| 12 | IL-10 | PE/DAZZLE | BioLegend | 505034 | JES5-16E3 |
| 13 | TNF-alpha | BV510 | BD Biosciences | 563386 | MP6-XT22 |
| 14 | IL-12p70 | V450 | BD Biosciences | 561456 | C15.6 |

### Animals, Wound Model, and Treatment

Equal numbers of male and female mice were used. Eight-week-old BALB/c mice were anesthetized with 120 mg/kg ketamine and 6 mg/kg xylazine by intraperitoneal injection prior to wounding. The dorsal surface was shaved with an electric clipper and prepped using chlorhexidine gluconate and alcohol swabbing in series on the surgical site. Five-millimeter (5 mm) biopsy punches were used to create middorsal full-thickness wounds by excising epidermis and dermis, including the panniculus carnosus. Immediately after the surgery on day 0, the wounds were topically treated with 10 μL of PBS containing 1 mg of soluble aKG, 2 mg of PaKG MPs, or no microparticle control (PBS only). A donut-shaped splint with an inner diameter of 5 mm prepared from a 0.5 mm-thick silicone sheet and covered on one side with Tegaderm (3M) was placed so that the wound was centered within the splint. An immediate-bonding adhesive. was used to fix the splint to the skin followed by interrupted 4-0 nylon sutures to ensure position. In order to prevent contraction of the wounds, silicone splint was used allowing wounds to heal through granulation and re-epithelialization. The mice were recovered on a heating pad until fully mobile. The mice were housed individually to prevent splint removal.

### Wound Area Image Analysis

Each wound site was digitally photographed everyday post-wounding, and wound areas were determined on photographs using ImageJ. The time-dependent wound areas were normalized to Day 0 wound area. Changes in wound areas were expressed as the proportion of the initial wound areas. All wound area measurements and plots are displayed as mean±standard error of mean (SEM) from six independent experiments.

### Ultimate Tensile Strength Measurements

Rectangular sections of the skin around the wound area (20×0.5 mm, measured by calipers after underlying fascia removal) were excised at days 10 post wounding. Skin samples were stretched until failure at a rate of 2 mm/sec using a TA.XTPlus texture analyzer. Ultimate tensile strength (UTS) was determined from the maximum force of the tissue prior to failure, where the maximum force (F) and area of the tissue sample (A) determined the ultimate tensile strength (σ, kPa) of the sutured skin (σ=F/A). The tensile strength of intact skin (with no incision) was also tested for comparison. All tensile strengths are displayed as mean±standard error of mean (SEM) from four independent experiments. Tensile strength recovery for skin samples were calculated as a difference between ultimate tensile strength for each group after healing from native skin strength, with the difference then converted to a percentage of the native skin.

### Statistics

Data are expressed as mean±standard error. Comparison between two groups was performed using Student's t-test. Comparisons between multiple treatment groups were performed using one-way ANOVA, followed by Bonferroni multiple comparisons, and p-values ≤0.05 was considered statistically significant. Statistical tests were performed using GraphPad Prism Software 6.0.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A particle comprising a polymer of a metabolite.

2. The particle of claim 1, wherein the metabolite comprises a phosphate group or a carboxylic acid group.

3. The particle of claim 1, wherein the metabolite is selected from the group consisting of α-ketoglutarate, succinic acid, Fructose, 1, 6 biphosphate (F16BP), fructose 6 phosphate, and phosphoenol pyruvic acid, and ribose 6 phosphate.

4. The particle of claim 3, wherein the metabolite is α-ketoglutarate.

5. The particle of claim 4, wherein the polymer of α-ketoglutarate comprises a structure of formula (I): 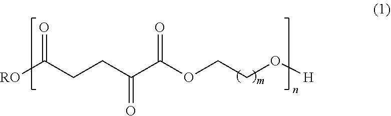
wherein R is a group selected from the group consisting of hydrogen, halogen, alkyl, aryl, heteroaryl, cycloalkyl, heterocyclyl, and combinations thereof; m is an integer from 1 to 30; and n is an integer greater than or equal to 1.

6. (canceled)

7. The particle of claim 1, wherein the particle has an average diameter of about 100 nanometer to 1 mm.

8. The particle of claim 1, wherein the particle has a molecular weight of about 1 kDa to about 25 kDa.

9. The particle of claim 1, wherein the particle encapsulates an active agent.

10. A biomaterial, wherein the biomaterial is coated with the composition of claim 1.

11. (canceled)

12. (canceled)

13. (canceled)

14. (canceled)

15. A method of modulating the intracellular metabolite profile of an immune cell, the method comprising administering the particle of claim 1 to the subject, wherein the particle delivers the metabolite to the subject and modulates the intracellular metabolite profile of one or more immune cells in the subject.

16. The method of claim 15, wherein the method modulates at least one pathway of the immune cell selected from the group consisting of: the glutamate pathway, the Krebs cycle pathway, the glycolysis pathway, and the arginine pathway.

17. The method of claim 16, wherein the method increases one or more of L-glutamate, 4-aminobutanoate asparagine, aspartate, acetyl-ornithine and L-citrulline in the immune cell.

18. (canceled)

19. (canceled)

20. (canceled)

21. (canceled)

22. A method of modulating immune response in a subject, the method comprising administering the particle of claim 1 to the subject, wherein the particle delivers the metabolite to the subject.

23. The method of claim 22, wherein the method decreases pro-inflammatory T cell responses in the subject or rescues immune cells against metabolic exhaustion.

24. (canceled)

25. A method of treating a disease or disorder in a subject, wherein disease or disorder is associated with increased or decreased immune activation, the method comprising administering the particle of claim 1 to the subject, wherein the particle delivers the metabolite to the subject.

26. A method of forming a metabolite-based polymer, the method comprising: forming a mixture of the metabolite comprising a carboxylic group and a diol compound of formula (A): 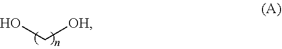
wherein n is an integer from 2 to 30.

27. The method of claim 26, wherein the method further comprises heating the mixture at about 35° C. to about 200° C.

28. The method of claim 27, wherein the mixture is heated for about 30 minutes to about 72 hours.

29. (canceled)

30. The method of claim 26 wherein the mixture further comprises SnCl₂

31. A polymer made by the method of claim 26.
